(19) United States
(12) Patent Application Publication
(10) Pub. No.: US 2012/0213855 A1
(43) Pub. Date: Aug. 23, 2012

(54) DOSAGE FORMS FOR WEAKLY IONIZABLE COMPOUNDS

(75) Inventors: Vikas AGARWAL, Plymouth, MN (US); Sami M. NAZZAL, Monroe, LA (US)

(73) Assignee: CIMA LABS INC., Eden Prairie, MN (US)

(21) Appl. No.: 13/029,235

(22) Filed: Feb. 17, 2011

(51) Int. Cl. A61K 9/18; A61K 9/107; A61P 29/00; A61P 9/12; A61P 11/06; A61P 9/00; A61P 9/10; A61K 31/5415; A61P 19/02

(52) U.S. Cl. 424/490; 514/226.5; 424/400

(57) ABSTRACT

This disclosure relates to dosage forms (e.g., solid dosage forms) comprising a drug-containing emulsion, a drug-containing microemulsion, a self-emulsifying oil composition, or a self-microemulsifying oil composition, wherein each comprises a weakly ionizable drug and a pH modifier. Also provided are pharmaceutical dosage forms (e.g., solid dosage forms) and methods of preparing the same.

### CROSS-REFERENCE TO RELATED APPLICATIONS

This application claims priority to PCT Application No. PCT/US2009/056362, filed Sep. 9, 2009, now published, which claims priority to U.S. Provisional Patent Application No. 61/161,992, filed Mar. 20, 2009, which claims the benefit to the filing date of U.S. Provisional Patent Application No. 61/095,493, filed Sep. 9, 2008, the disclosure of which is hereby incorporated herein.

### TECHNICAL FIELD

This disclosure relates to dosage forms comprising a drug-containing emulsion, a drug-containing microemulsion, a self-emulsifying oil composition, or a self-microemulsifying oil composition, wherein each comprises a weakly ionizable drug and a pH modifier. Also provided are pharmaceutical dosage forms, such as solid dosage forms, and methods of preparing the same.

### BACKGROUND

Certain drugs present problems in balancing the desire for a convenient oral dosing format and the necessary bioavailability. With some drugs, absorption of an orally administered dose could be as little as 30%, or less. Such poorly absorbed drugs often display large inter- and intra-subject variability in bioavailability.

The absorption rate of most drugs depends on two factors: (1) the dissolution of the drug in physiological fluids and (2) the absorption process itself, i.e., the process by which a drug in solution enters the cells at the absorption site and finally enters general circulation. Many drugs are absorbed by passive diffusion, i.e., a spontaneous migration of drug molecules from a region of high concentration to a region of low concentration. Other drugs are absorbed by active transportation which involves the expenditure of energy by the body. Typically, for solid orally administered drugs, absorbed actively or passively, dissolution of the drug is the first step in the absorption process.

### SUMMARY

This disclosure relates to dosage forms (e.g., solid dosage forms) comprising a drug-containing emulsion, a drug-containing microemulsion, a self-emulsifying oil composition, or a self-microemulsifying oil composition, wherein each comprises a weakly ionizable drug and a pH modifier. Also provided are pharmaceutical dosage forms and methods of preparing the same.

Provided herein is a dosage form including: (a) a drug-containing emulsion having globules of diameter greater than 100 nm, wherein the drug-containing emulsion comprises a weakly ionizable drug and a pH modifier, wherein the pH modifier increases the solubility of the weakly ionizable drug in an aqueous phase as compared to the solubility of the weakly ionizable drug in the absence of the pH modifier, and wherein the weakly ionizable drug has a pKa of from about 1 to about 14; and (b) a solid particle adsorbent, wherein the drug-containing emulsion is adsorbed to the solid particle adsorbent to form a free-flowing compressible powder.

In some embodiments, the emulsion globules can have diameters of from about 120 nm to about 70 μm. In some embodiments, the drug-containing emulsion includes from about 1% to about 50% by weight of the weakly ionizable drug based on the total weight of both phases of the emulsion. In some embodiments, the drug-containing emulsion can be an oil-in-water emulsion or a water-in-oil emulsion.

Also provided herein is a dosage form including: (a) a drug-containing microemulsion having globules of diameter less than 100 nm, wherein the drug-containing microemulsion comprises a weakly ionizable drug and a pH modifier, wherein the pH modifier increases the solubility of the weakly ionizable drug in an aqueous phase as compared to the solubility of the weakly ionizable drug in the absence of the pH modifier, and wherein the weakly ionizable drug has a pKa of from about 1 to about 14; and (b) a solid particle adsorbent, wherein the drug-containing microemulsion is adsorbed to the solid particle adsorbent to form a free-flowing compressible powder.

In some embodiments, the drug-containing microemulsion comprises from about 1% to about 50% by weight of the weakly ionizable drug based on the total weight of both phases of the microemulsion. In some embodiments, the drug-containing microemulsion can be an oil-in-water emulsion or a water-in-oil emulsion.

Further provided herein is a dosage form including: (a) a self-emulsifying oil composition comprising one or more oils, a weakly ionizable drug and a pH modifier, wherein the self-emulsifying oil composition forms a drug-containing emulsion having globules of diameter greater than 100 nm when exposed to an aqueous phase, wherein the pH modifier increases the solubility of the weakly ionizable drug in an aqueous phase as compared to the solubility of the weakly ionizable drug in the absence of the pH modifier, and wherein the weakly ionizable drug has a pKa of from about 1 to about 14; and (b) a solid particle adsorbent, wherein the self-emulsifying oil composition is adsorbed to the solid particle adsorbent to form a free-flowing compressible powder.

In some embodiments, the self-emulsifying oil composition comprises from about 1% to about 50% by weight of the weakly ionizable drug based on the total weight of the oil composition. In some embodiments, the emulsion globules can have diameters of from about 120 nm to about 70 μm.

This disclosure also provides a dosage form including: (a) a self-microemulsifying oil composition comprising one or more oils, a weakly ionizable drug and a pH modifier, wherein the self-microemulsifying oil composition forms a drug-containing microemulsion having globules of diameter less than 100 nm when exposed to an aqueous phase, wherein the pH modifier increases the solubility of the weakly ionizable drug in an aqueous phase as compared to the solubility of the weakly ionizable drug in the absence of the pH modifier, and wherein the weakly ionizable drug has a pKa of from about 1 to about 14; and (b) a solid particle adsorbent, wherein the self-microemulsifying oil composition is adsorbed to the solid particle adsorbent to form a free-flowing compressible powder.

In some embodiments, the self-microemulsifying oil composition includes from about 1% to about 50% by weight of the weakly ionizable drug based on the total weight of the oil composition.

In some embodiments, the dosage forms described herein can include a solid particle selected from the group consisting of kaolin; bentonite; hectorite; colloidal magnesium aluminum silicate; silicon dioxide; magnesium trisilicate; aluminum hydroxide; magnesium hydroxide; magnesium oxide; and talc.

In some embodiments, the weakly ionizable drug can be selected from the group consisting of meloxicam; atropine; chloramphenicol; chlorothiazide; chlorpromazine; cimetidine; diazepam; diltiazem; diphenhydramine; disopyramide; flufenamic acid; furosemide; haloperidol; imipramine; lidocaine; phenobarbital; phenyloin; procainamide; propafenone; propranolol; tetracaine; trimethoprim; and verapamil. In some embodiments, the weakly ionizable drug is meloxicam.

In some embodiments, the weakly ionizable drug can be poorly soluble. In some embodiments, the poorly soluble drug can exhibit an absolute bioavailability of greater than about 30%.

In some embodiments, the pH modifier can be selected from the group consisting of: sodium carbonate; potassium carbonate; magnesium carbonate; sodium bicarbonate; potassium bicarbonate; disodium hydrogen phosphate; sodium dihydrogen phosphate; dipotassium hydrogen phosphate; potassium dihydrogen phosphate; tris(hydroxymethyl)aminomethane; citric acid; tartaric acid; amalic acid; fumaric acid; adipic acid; and succinic acid. In some embodiments, the pH modifier is tris(hydroxymethyl)aminomethane.

Provided herein is a dosage form including: (a) a drug-containing emulsion having globules of diameter greater than 100 nm, wherein the drug-containing emulsion comprises meloxicam and a pH modifier, and wherein the pH modifier increases the solubility of meloxicam in an aqueous phase as compared to the solubility of meloxicam in the absence of the pH modifier; and (b) a solid particle adsorbent, wherein the drug-containing emulsion is adsorbed to the solid particle adsorbent to form a free-flowing compressible powder.

Also provided herein is a dosage form including: (a) a drug-containing microemulsion having globules of diameter less than 100 nm, wherein the drug-containing microemulsion comprises meloxicam and a pH modifier, and wherein the pH modifier increases the solubility of meloxicam in an aqueous phase as compared to the solubility of meloxicam in the absence of the pH modifier; and (b) a solid particle adsorbent, wherein the drug-containing microemulsion is adsorbed to the solid particle adsorbent to form a free-flowing compressible powder.

Further provided herein is a dosage form including: (a) a self-emulsifying oil composition comprising one or more oils, meloxicam and a pH modifier, wherein the self-emulsifying oil composition forms a drug-containing emulsion having globules of diameter greater than 100 nm when exposed to an aqueous phase, and wherein the pH modifier increases the solubility of meloxicam in an aqueous phase as compared to the solubility of meloxicam in the absence of the pH modifier; and (b) a solid particle adsorbent, wherein the self-emulsifying oil composition is adsorbed to the solid particle adsorbent to form a free-flowing compressible powder.

This disclosure also provides a dosage form including (a) a self-microemulsifying oil composition comprising one or more oils, meloxicam and a pH modifier, wherein the self-microemulsifying oil composition forms a drug-containing microemulsion having globules of diameter less than 100 nm when exposed to an aqueous phase, and wherein the pH modifier increases the solubility of meloxicam in an aqueous phase as compared to the solubility of meloxicam in the absence of the pH modifier; and (b) a solid particle adsorbent, wherein the self-microemulsifying oil composition is adsorbed to the solid particle adsorbent to form a free-flowing compressible powder.

A dosage form, as described herein, comprising meloxicam, can in some embodiments, upon administration achieve a shorter Tₘₐₓ compared to a composition comprising meloxicam that is not an emulsion, microemulsion, self-emulsifying oil composition, self-microemulsifying oil composition, that does not comprise the pH modifier, and that is administered by the same route. In some embodiments, the Tₘₐₓ can range from about 94 to about 142 minutes. In some embodiments, the Tₘₐₓ can range from about 116 to about 120 minutes.

In some embodiments, a dosage form comprising meloxicam can dissolve faster in water compared to a composition comprising meloxicam that is not an emulsion, microemulsion, self-emulsifying oil composition, self-microemulsifying oil composition, that does not comprise the pH modifier, and that is administered by the same route. In some embodiments, from about 75 to about 100% of 15 mg of the dosage form dissolves in about 15 minutes in 900 mL water. In some embodiments, about 90% of 15 mg of the dosage form dissolves in about 15 minutes in 900 mL water.

Provided herein is a drug-containing emulsion having globules of diameter greater than 100 nm, wherein the drug-containing emulsion comprises a weakly ionizable drug and a pH modifier, wherein the pH modifier increases the solubility of the weakly ionizable drug in an aqueous phase as compared to the solubility of the weakly ionizable drug in the absence of the pH modifier, and wherein the weakly ionizable drug has a pKa of from about 1 to about 14.

Also provided herein is a drug-containing microemulsion having globules of diameter less than 100 nm, wherein the drug-containing microemulsion comprises a weakly ionizable drug and a pH modifier, wherein the pH modifier increases the solubility of the weakly ionizable drug in an aqueous phase as compared to the solubility of the weakly ionizable drug in the absence of the pH modifier, and wherein the weakly ionizable drug has a pKa of from about 1 to about 14.

Further provided herein is a self-emulsifying oil composition comprising one or more oils, a weakly ionizable drug and a pH modifier, wherein the self-emulsifying oil composition forms a drug-containing emulsion having globules of diameter greater than 100 nm when exposed to an aqueous phase, wherein the pH modifier increases the solubility of the weakly ionizable drug in an aqueous phase as compared to the solubility of the weakly ionizable drug in the absence of the pH modifier, and wherein the weakly ionizable drug has a pKa of from about 1 to about 14.

This disclosure also provides a self-microemulsifying oil composition comprising one or more oils, a weakly ionizable drug and a pH modifier, wherein the self-microemulsifying oil composition forms a drug-containing microemulsion having globules of diameter less than 100 nm when exposed to an aqueous phase, wherein the pH modifier increases the solubility of the weakly ionizable drug in an aqueous phase as compared to the solubility of the weakly ionizable drug in the absence of the pH modifier, and wherein the weakly ionizable drug has a pKa of from about 1 to about 14.

A solid dosage form for oral administration of a therapeutically effective amount of a drug is provided, the solid dosage form including one of the dosage forms as described herein. In some embodiments, the solid dosage form is a tablet. In some embodiments, the solid dosage form also includes at least one effervescent agent or effervescent system. In some embodiments, the solid dosage form also includes at least one disintegration agent; wherein the disintegration agent causes rapid dispersion of the drug-containing emulsion to a target area following oral administration.

Further provided herein is a method for preparing a dosage form as described herein. In some embodiments, the method includes: (a) preparing a drug-containing emulsion or microemulsion including a weakly ionizable drug, wherein the weakly ionizable drug has a pKa ranging from about 1 to about 14, and a pH modifier; and (b) converting the drug-containing emulsion or microemulsion into a free-flowing compressible powder by admixing the drug-containing emulsion with a solid particle adsorbent. In some embodiments, the method includes: (a) preparing a drug-containing self-emulsifying or self-microemulsifying oil composition comprising one or more oils, a weakly ionizable drug, wherein the weakly ionizable drug has a pKa ranging from about 1 to about 14, and a pH modifier; and (b) converting the drug-containing self-emulsifying oil composition into a free-flowing compressible powder by admixing the drug-containing self-emulsifying drug delivery system with a solid particle adsorbent.

A method for preparing a solid dosage form for oral administration of a therapeutically effective amount of a drug is also provided. The method can include, preparing a dosage form as described above, and compressing the free-flowing, compressible powder into a solid dosage form.

Also provided herein is a method of administering a drug to a mammal including the steps of preparing a dosage form as described herein and orally administering the free-flowing compressible powder to the mammal.

Further provided herein is a method of treating pain in a mammal, the method including administering to the mammal a dosage form including meloxicam as described herein. In some embodiments, the pain can include lower back pain, acute pain, and pain associated with inflammation.

This disclosure also provides a method of treating arthritis in a mammal, the method comprising administering to the mammal a dosage form including meloxicam as described herein. In some embodiments, the arthritis can include osteoarthritis, rheumatoid arthritis, and juvenile rheumatoid arthritis.

Also provided herein is a method of treating one or more of ankylosing spondylitis, fever, primary dysmenorrheal, pyrexia, asthma, bone resorption, cardiovascular disease, nephrotoxicity, atherosclerosis, or hypotension, the method comprising administering to the mammal a dosage form including meloxicam as described herein.

Further provided herein is a composition having from about 20% to about 40% by weight of a co-solvent; from about 4% to about 9% by weight of a pH modifier; from about 9% to about 18% by weight of purified water; from about 28% to about 56% by weight of a surfactant; and from about 5% to about 10% by weight of a weakly ionizable drug. In some embodiments, the composition includes about 30% by weight of a co-solvent; about 6.67% by weight of a pH modifier; about 13.33% by weight of purified water; about 42% by weight of a surfactant; and about 8% by weight of a weakly ionizable drug.

In some embodiments, the composition is component of a drug-containing emulsion, drug-containing microemulsion, self-emulsifying oil composition, or self-microemulsifying oil composition.

In some embodiments, the composition further comprises a solid particle adsorbent. In some cases, the solid particle adsorbent is present in a ratio from about 1:5 to about 5:1 by weight of the composition.

Also provided herein is a composition having from about 10% to about 20% by weight of a co-solvent; from about 2% to about 5% by weight of a pH modifier; from about 4% to about 9% by weight of purified water; from about 14% to about 28% by weight of a surfactant; from about 2% to about 6% by weight of a weakly ionizable drug; and from about 35% to about 65% by weight of a solid particle adsorbent. In some embodiments, the composition includes about 15% by weight of a co-solvent; about 3.33% by weight of a pH modifier; about 6.67% by weight of purified water; about 21% by weight of a surfactant; about 4% by weight of a weakly ionizable drug; and about 50% by weight of a solid particle adsorbent.

In some embodiments, the weakly ionizable drug can be selected from the group consisting of meloxicam; atropine; chloramphenicol; chlorothiazide; chlorpromazine; cimetidine; diazepam; diltiazem; diphenhydramine; disopyramide; flufenamic acid; furosemide; haloperidol; imipramine; lidocaine; phenobarbital; phenyloin; procainamide; propafenone; propranolol; tetracaine; trimethoprim; verapamil; and mixtures thereof. In some embodiments, the weakly ionizable drug is meloxicam.

Further provided herein is a composition having from about 20% to about 40% by weight of a co-solvent; from about 4% to about 9% by weight of a pH modifier; from about 9% to about 18% by weight of purified water; from about 28% to about 56% by weight of a surfactant; and from about 5% to about 10% by weight of meloxicam. In some embodiments, the composition includes about 30% by weight of a co-solvent; about 6.67% by weight of a pH modifier; about 13.33% by weight of purified water; about 42% by weight of a surfactant; and about 8% by weight of meloxicam. In some embodiments, the composition includes about 3% by weight of Captex 355®; about 5% by weight Capmul MCM®; about 2% by weight PVP K12; about 20% by weight Propylene Glycol; about 6.67% by weight aqueous tris(hydroxymethyl)aminomethane; about 13.33% by weight purified water; about 42% by weight Tween 80®; and about 8% by weight meloxicam.

In some embodiments, the composition is component of a drug-containing emulsion, drug-containing microemulsion, self-emulsifying oil composition, or self-microemulsifying oil composition.

In some embodiments, the composition further includes a solid particle adsorbent. In some embodiments, the solid particle adsorbent is present in a ratio from about 1:5 to about 5:1 by weight of the composition. In some embodiments, the ratio is about 1:1.

Also provided herein is a composition having from about 10% to about 20% by weight of a co-solvent; from about 2% to about 5% by weight of a pH modifier; from about 4% to about 9% by weight of purified water; from about 14% to about 28% by weight of a surfactant; from about 2% to about 6% by weight of meloxicam; and from about 35% to about 65% by weight of a solid particle adsorbent. In some embodiments, the composition includes about 15% by weight of a co-solvent; about 3.33% by weight of a pH modifier; about 6.67% by weight purified water; about 21% by weight of a surfactant; about 4% by weight of meloxicam; and about 50% by weight of a solid particle adsorbent.

In some embodiments, the composition includes about 1.5% by weight of Captex 355®; about 2.5% by weight Capmul MCM®; about 1% by weight PVP K12; about 10% by weight Propylene Glycol; about 3.33% by weight aqueous tris(hydroxymethyl)aminomethane; about 6.67% by weight purified water; about 21.25% by weight Tween 80®; about 4% by weight meloxicam; and about 50% by weight silicon dioxide.

In the compositions described above, the co-solvent can be selected from the group consisting of Captex-355® (Glyceryl Tricaprylate/Caprate); Labrasol® (Caprylocaproyl Macrogol Glycerides); Labrafil M1944® (Oleoyl Macrogol Glycerides); Lauroglycol 90® (Propylene Glycol Monolaurate); Tween 80® (Polyoxyethylene (20) Sorbitan Monoleate); PEG 400 (Polyethylene Glycol); Capmul MCM® (Medium chain Mono- and Diglycerides); Capmul PG8® (Propylene Glycol Monocaprylate); Plurol Oleique® (polyglyceryl Oleate); Span 80® (Sorbitan Monoleate); Cremophor EL® (Polyoxyl 35 Castor Oil); Phosal 53MCT® (53% Phosphatidylcholine in Medium Chain Triglycerides); Corn oil; Oleic acid; Triethyl Citrate; Ethanol; Water; Acconon CC-6® (Polyoxyethylene 6 Caprylic/Capric Glycerides); PVP (Polyvinylpyrrolidone) K12; PVP K17; PVP K30; PVP K90; propylene glycol; and mixtures thereof. In some embodiments, the co-solvent can be selected from the group consisting of Captex 355®; Capmul MCM®; PVP K12®; Propylene glycol; and mixtures thereof. In some embodiments, the co-solvent can be about 3% Captex 355®; 5% Capmul MCM®; about 2% PVP K12®; and about 20% Propylene glycol.

In some embodiments, the pH modifier can be selected from the group consisting of sodium carbonate; potassium carbonate; magnesium carbonate; sodium bicarbonate; potassium bicarbonate; disodium hydrogen phosphate; sodium dihydrogen phosphate; dipotassium hydrogen phosphate; potassium dihydrogen phosphate; tris(hydroxymethyl)aminomethane; citric acid; tartaric acid; amalic acid; fumeric acid; adipic acid; succinic acid; and mixtures thereof. In some embodiments, the pH modifier is tris(hydroxymethyl)aminomethane.

In some embodiments, the surfactant can be selected from the group consisting of Tween 85® (Polyoxyethylene (20) Sorbitan Trioleate); Tween 80® (Polyoxyethylene (20) Sorbitan Monoleate); Tween 20 (Polyoxyethylene (20) Sorbitan Monolaurate); Labrasol® (Caprylocaproyl Macrogol Glycerides); Cremophor EL® (Polyoxyl 35 Castor Oil); Triton X-100® (Octylphenol Ethoxylate); Oleic acid; Isopropyl Myristate; Ethyl Oleate; and mixtures thereof. In some embodiments, the surfactant is Tween 80®.

In some embodiments, the solid particle adsorbent is selected from the group consisting of kaolin, bentonite, hectorite, colloidal magnesium aluminum silicate, silicon dioxide, magnesium trisilicate, aluminum hydroxide, magnesium hydroxide, magnesium oxide, and talc. In some embodiments, the solid particle adsorbent is silicon dioxide.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows a dissolution profile of meloxicam, a meloxicam self-emulsifying oil composition, and a meloxicam self-emulsifying oil composition adsorbed onto two silicon dioxides and a MgAl silicate powder.

FIG. 2 shows the pharmacokinetic data from an in vivo dog study for three formulations of meloxicam: 1) self-microemulsifying oil composition adsorbed onto silicon dioxide particles (MicroSolv); 2) self-microemulsifying oil composition (liquid SMEDDS); and Mobic (tablet).

FIG. 3 details the dissolution profile for a self-microemulsifying oil composition adsorbed onto silicon dioxide particles.

FIG. 4 shows the pharmacokinetic data from an in vivo human study for three orally administered formulations of meloxicam: A) self-microemulsifying oil composition adsorbed onto silicon dioxide particles; B) self-microemulsifying oil composition; and C) Mobic (tablet).

FIG. 5 demonstrates dissolution of a self-microemulsifying oil composition of meloxicam adsorbed onto silicon dioxide particles in a variety of aqueous media.

FIG. 6 demonstrates dissolution of a meloxicam tablet (Mobic) in a variety of aqueous media.

### DETAILED DESCRIPTION

This disclosure relates to dosage forms (e.g., solid dosage forms) comprising a drug-containing emulsion, a drug-containing microemulsion, a self-emulsifying oil composition, or a self-microemulsifying oil composition, wherein each comprises a weakly ionizable drug and a pH modifier. Also provided are pharmaceutical dosage forms and methods of preparing the same.

A dosage form can be provided in the form of a drug-containing emulsion, a drug-containing microemulsion, a self-emulsifying oil composition, or a self-microemulsifying oil composition. A dosage form can be provided in the form of a free-flowing, compressible powder, comprising one or more of a drug-containing emulsion, a drug-containing microemulsion, a self-emulsifying oil composition, or a self-microemulsifying oil composition, and a solid particle adsorbent. The drug-containing emulsion, drug-containing microemulsion, self-emulsifying oil composition or self-microemulsifying oil composition can be adsorbed on the solid particle adsorbent and can form a free-flowing, compressible powder.

In some embodiments, a dosage form can be provided as a liquid formulation, comprising: one of a drug-containing emulsion, a drug-containing microemulsion, a self-emulsifying oil composition, or a self-microemulsifying oil composition.

A drug-containing emulsion and a drug-containing microemulsion each comprise an emulsion or microemulsion, respectively, comprising a weakly ionizable drug and a pH modifier. A self-emulsifying oil composition and a self-microemulsifying oil composition each comprise one or more oils, a weakly ionizable drug, and a pH modifier. A drug-containing emulsion, microemulsion, self-emulsifying oil composition, or self-microemulsifying oil composition can include one or more emulsifying agents, anti-oxidants, or preservatives, or mixtures thereof, as described further below.

An emulsion, as used herein, is a system of two immiscible liquid phases. One of the two phases (the internal phase) is distributed as droplets or globules throughout the second phase (the external or continuous phase). Emulsions can include oil-in-water (o/w) emulsions, in which a less polar liquid commonly referred to as an oil is in the internal phase; and water-in-oil (w/o) emulsions, in which an aqueous or other relatively polar liquid is in the internal phase. Typically, an emulsion has globule diameters of greater than about 100 nm. In some embodiments, emulsion globule diameters range from about 120 nm to about 70 μm. See, e.g., U.S. Pat. No. 6,692,771. Emulsions can also be classified as fine emulsions with globule diameters of less than about 5 μm and coarse emulsions with globule diameters greater than about 5 μm.

A microemulsion is an optically isotropic and thermodynamically or kinetically stable system. Microemulsions are composed of an oil phase and an aqueous phase. In some embodiments, a microemulsion can also contain an emulsifying agent. Typically, a microemulsion has globule diameters of less than about 100 nm (e.g., from about 30 to about 60 nm). See, e.g., U.S. Pat. No. 6,280,770.

The disclosure also provides the use of self-emulsifying and self-microemulsifying oil compositions which include all the components of an o/w emulsion or microemulsion (e.g., one or more of an oil, emulsifying agent, antioxidant, and preservative) with the exception of water. See, e.g., U.S. Pat. No. 5,444,041. In some cases, a self-emulsifying and self-microemulsifying oil composition can be a self-emulsifying drug delivery system (SEDDS) or a self-microemulsifying drug delivery system (SMEDDS), respectively. In some embodiments, a solid dosage form having a self-emulsifying or self-microemulsifying oil composition can be prepared by adsorbing the self-emulsifying or self-microemulsifying oil composition to an adsorbent powder. After administration of the solid dosage form containing a self-emulsifying or self-microemulsifying oil composition, an emulsion or microemulsion forms in vivo upon admixing with the body fluids.

The oil phase in an emulsion or microemulsion can be any nontoxic oil, which includes, but is not limited to mono-, di- and triglycerides, fatty acids and their esters, and ethers and esters of propylene glycol or other polyols. Fatty acids and esters (used as such or where they form part of a glyceride) may be short chain, medium chain, or long chain. As used herein, medium chain represents a hydrocarbon chain of C₈ to C₁₂, short chain is a hydrocarbon chain of less than C₈, and long chain refers to a hydrocarbon chain of more than C₁₂.

The oil phase may be of vegetable or animal origin. The oil phase may also be synthetic or semisynthetic, and nontoxic to the subject. The oils include, but are not limited to, natural oils, such as cottonseed oil, soybean oil, sunflower oil; canola oil; CAPTEX® (various grades propylene glycol esters such as propylene glycol didecanoate; and glycerol esters such as glyceryl tricaprylate/caprate); MIGLYOL® (caprylic/capric acid triglycerides; caprylic/capric/linoleic acid triglycerides; caprylic/capric/succinic acid triglycerides; or propylene glycol diester of caprylic/capric acid and admixtures with other agents); and CAPMUL® (available in different grades, e.g. Capmul MCM, which is mainly mono- and di-esters of glycerol and of propylene glycol, such as glyceryl monooleate and propylene glycol monocaprilate. Another grade consists of polyethylene glycol glyceryl monostearate.).

The water phase in an emulsion or microemulsion can be, for example, water, aqueous solutions, alcohols, and alcohol solutions.

The formation of emulsions and microemulsions can require an emulsifying agent. As used herein, any nontoxic emulsifying agents may be used. This includes, but is not limited to, various grades of the following commercial products: MYVACET® (distilled acetylated monoglyceride emulsifers); ARLACEL® (mainly sorbitan esters); TWEEN® (polyoxyethylene sorbitan esters); CENTROPHASE® (fluid lecithins); CREMOPHOR® (polyoxyl castor oil derivatives; or macrogol ethers; or macrogol esters); LABRAFAC® (caprylic/capric triglyceride); LABRAFIL® (polyoxyethylated glycolysed glycerides); LABRASOL (mixture of mono-, di- and triglycerides and mono-and di-fatty esters of polyethylene glycol; the predominant fatty acids are C₈-C₁₀ caprylic/capric acids); MYVEROL®; and TAGAT® (polyethyleneglycol hydrogenated castor oil; or polyethyleneglycol glyceryl esters); lecithin; cholesterol and proteins such as casein. In some embodiments, multiple emulsifying agents can be used to maintain the internal phase distributed as globules throughout the external phase and to retard coalescence of the globules into larger drops. In this way, the two phases can be kept in relative stability for a longer period of time.

In pharmaceutical emulsions and microemulsions, one or both phases can include a drug or a solution of one or more drugs. In some embodiments, either or both the water and the oil phases can contain a drug at the same time and those drugs may be the same or different. Any two immiscible liquids that are non-toxic and compatible with the part of the mammalian body to which they are to be applied may be used.

An oil phase, aqueous phase, and optionally, an emulsifying agent can be used in a wide range of ratios to make the emulsions or microemulsions. For example, oil-in-water emulsions and microemulsions can contain at least about 25% of water by weight (e.g., from about 60% to about 98% by weight and from about 70% to about 90% by weight). The oil phase in an o/w emulsion or microemulsion can be at least about 1% of the emulsion or microemulsion by weight (e.g., from about 5% to about 69% by weight and from about 8% to about 40% by weight), wherein the active drug ingredient is included in the weight of the oil phase. The emulsifying agent in the emulsion or microemulsion can be at least about 0.5% by weight (e.g., from about 2% to about 25% by weight and from about 5% to about 10% by weight).

Water-in-oil emulsions and microemulsions can contain at least about 25% oil phase by weight of the w/o emulsion or microemulsion (e.g., from about 40% to about 98% and from about 50% to about 95%). The water phase in w/o emulsions and microemulsions can be at least about 1% of the w/o emulsion or microemulsion by weight (e.g., from about 2% to about 55% and from about 5% to about 30%) wherein the active drug ingredient is included in the weight of the water phase. The emulsifying agent in w/o emulsions and microemulsions can be at least about 0.5% of the w/o emulsion or microemulsion by weight (e.g., from about 2% to about 20% and from about 4% to about 10%).

Emulsifying agents or combinations of agents are used for o/w or w/o formulations in accordance with the HLB (hydrophile-lipophile balance) system. A w/o emulsion or microemulsion requires low HLB emulsifying agents (HLB value approximately 1 to 7) and an o/w system requires higher HLB emulsifying agents (HLB value approximately 11 to 18). In general, the type of emulsifying agent used and the relative proportions of oil and water determine whether the emulsion or microemulsion is a w/o or o/w emulsion or microemulsion. This refers to the emulsion or microemulsion upon formation. Some emulsions or microemulsions may invert when added to a large volume of the internal phase. For example, an emulsion or microemulsion that is prepared as a w/o emulsion or microemulsion, upon consumption by the patient may invert to an o/w emulsion or microemulsion in the patient's stomach.

Typically, an emulsion can be prepared by mixing the components of the emulsion (e.g., the oil phase, water phase, emulsifying agent, etc.) in a propeller mixer, a turbine mixer or other high shear mixer and stirring the mixture vigorously.

Microemulsions can be prepared, sometimes spontaneously, by combining the components in the correct proportions. In some embodiments, light mixing with a propeller mixer can be used. Unless ingredients which are solid at room temperature are used, there is no need for the use of heat in the preparation of microemulsions. Microemulsions can have a long shelf life, a low viscosity for easy transport and mixing, and, being translucent, can be monitored spectroscopically.

To prepare a dosage form, an adsorbent can be placed in the bowl of a suitable mixer, such as a planetary mixer, and the drug-containing emulsion, drug-containing microemulsion, self-emulsifying oil composition or self-microemulsifying oil composition can be added slowly with mixing. The rate of addition should not be so fast as to form clumps of wet material or, alternately, areas that are much more wet than other areas of powder. If powder clumps or wet portions of power are encountered, the rate of addition should be reduced or temporarily stopped until the wet portion of powder is well distributed throughout the bulk of the powder.

Any mixer that affords uniform mixing of powders with liquids can be used in the preparation of a dosage form as described herein. For example, a planetary mixer is well suited to this operation since it is equipped with a scraper bar that scrapes material off the sidewalls of the vessel. Overly wet material often accumulates on the sidewalls and would not have mixed well with the bulk powder, except for the action of the scraper bar. Mixers that have dead space should be avoided. A dead space, for purposes of the present discussion, is one in which powder can collect but in which space the powder is not subject to the mixing action of the apparatus. Mixers which have a secondary mixing action, in addition to the primary action, are preferred. The secondary mixer action may be a scraper (as in the planetary mixer) or an intensifier bar or other high shear component. Generally, the mixing action of the mixer should provide an intense mixing zone where high shear of the powder is experienced as well as an additional mixing action which moves all portions of the powder through the intense mixing zone, i.e. there should be a three dimensional shuffling of the bulk powder.

Addition of the drug-containing emulsion, drug-containing microemulsion, self-emulsifying oil composition or self-microemulsifying oil composition to the powder may be accomplished by the operator simply pouring the drug-containing emulsion, drug-containing microemulsion, self-emulsifying oil composition or self-microemulsifying oil composition into the mixer bowl (containing the powder) from a beaker or measuring cylinder, or other means such as spraying the liquid onto the powder at a controlled rate. In some embodiments, a peristaltic pump can be used to add liquid at a controlled rate. In some embodiments, a spray nozzle used in conjunction with a peristaltic pump can be used to provide a fine spray.

The present disclosure provides for weakly ionizable drugs formulated with a pH modifier. The drug may be combined with either the oil phase or the water phase depending on its solubility and other characteristics. A drug dissolved in one phase may partition into the other phase to some extent and this can affect the bioavailability of the drug.

Any weakly ionizable substance (drug) may be used in the drug-containing emulsion, drug-containing microemulsion, self-emulsifying oil composition or self-microemulsifying oil composition. Liquid drugs, drug solutions, small molecule drugs and nutritional supplements, such as vitamins and minerals, are all suitable for use.

As used herein, the phrase “small molecule” includes any inorganic chemical molecules, organic chemical molecules having a molecular weight of less than 3,000 Daltons.

Weakly ionizable drugs include meloxicam; atropine; chloramphenicol; chlorothiazide; chlorpromazine; cimetidine; diazepam; diltiazem; diphenhydramine; disopyramide; flufenamic acid; furosemide; haloperidol; imipramine; lidocaine; phenobarbital; phenyloin; procainamide; propafenone; propranolol; tetracaine; trimethoprim; and verapamil.

A weakly ionizable drug, as described herein, can have a pKa ranging from about 1 to about 14 (e.g., about 1 to about 13; about 1 to about 12; about 1 to about 11; about 1 to about 10; about 1 to about 9; about 1 to about 8; about 1 to about 7; about 1 to about 6; about 1 to about 5; about 1 to about 4; about 1 to about 3; about 1 to about 2; about 2 to about 14; about 3 to about 14; about 4 to about 14; about 5 to about 14; about 6 to about 14; about 7 to about 14; about 8 to about 14; about 9 to about 14; about 10 to about 14; about 11 to about 14; about 12 to about 14; about 13 to about 14; about 2 to about 13; about 3 to about 12; about 4 to about 10; about 5 to about 12; about 6 to about 9; and about 4 to about 8).

In some embodiments, the drug-containing emulsion, drug-containing microemulsion, self-emulsifying oil composition or self-microemulsifying oil composition contains from about 0.5% to about 60% of a weakly ionizable drug by weight of the total emulsion (both phases) or oil composition (e.g., from about 1% to about 55%; from about 3% to about 45%; from about 5% to about 40% from about 10% to about 35%; from about 15% to about 25%; and from about 6% to about 48%; from about 8% to about 52%; and from about 4% to about 36%). The weakly ionizable drug can be in the range of from about 2% to about 50% of the total weight of the drug-containing emulsion or drug-containing microemulsion (both phases) or the self-emulsifying or microemulsifying oil composition (e.g., from about 5% to about 40% and from about 10% to about 30%).

The drug-containing emulsion or microemulsion has a viscosity of between 1 cps and 400,000 cps (e.g., from about 400 cps to about 200,000 cps; from about 5,000 cps to about 150,000 cps; from about 10,000 cps to about 100,000 cps; from about 20,000 cps to about 80,000 cps; from about 30,000 cps to about 50,000 cps; and from about 500 cps to about 10,000 cps).

Any suitable and pharmaceutically acceptable pH modifier can be used in a drug-containing emulsion, drug-containing microemulsion, self-emulsifying oil composition or self-microemulsifying oil composition. In some embodiments, the selection of a pH modifier can be based on the characteristics of the drug, e.g., its pKa, solubility, and reactivity. A pH modifier is used with a weakly ionizable drug to increase the solubility of the weakly ionizable drug in an aqueous phase compared to the solubility of the weakly ionizable drug in the absence of the pH modifier. Non-limiting examples of pH modifiers include sodium carbonate; potassium carbonate; magnesium carbonate; sodium bicarbonate; potassium bicarbonate; disodium hydrogen phosphate; sodium dihydrogen phosphate; dipotassium hydrogen phosphate; potassium dihydrogen phosphate; tris(hydroxymethyl)aminomethane; citric acid; tartaric acid; amalic acid; fumeric acid; adipic acid; and succinic acid. In some embodiments, the pH modifier is tris(hydroxymethyl)aminomethane. Additional pH modifiers as described in U.S. patent application Ser. Nos. 09/302,105 and 09/327,814, hereby incorporated by reference herein, may also be used.

In some embodiments, the drug-containing emulsion, drug-containing microemulsion, self-emulsifying oil composition or self-microemulsifying oil composition contains from about 0.5% to about 60% of the pH modifier by weight of the total emulsion (both phases) or oil composition. The pH modifier can be in the range of from about 2% to about 50% of the total weight of the drug-containing emulsion or microemulsion (both phases) or the self-emulsifying or microemulsifying oil composition (e.g., from about 5% to about 40% and from about 10% to about 30%).

In some embodiments, a dosage form can also contain additional excipients, for example, preservatives, antioxidants, colors, flavors, and fragrances. Non-limiting examples of preservatives include methylparaben, propylparaben, benzoic acid, and cetylpyridinium chloride.

Emulsions, including drug-containing emulsions, have different characteristics as compared with, for example, microemulsions and drug-containing microemulsions. Generally, both emulsions and microemulsions consist of globules of one phase, e.g. water, in another phase, e.g. oil, wherein the emulsion globules have larger diameter than the microemulsion globules. Generally, emulsions have globules with mean diameters (the average diameter of all globules in the emulsion) larger than 0.1 μm or 100 nm (e.g., from about 0.16 μm to about 40 μm), while microemulsions contain globules having diameter of less than 0.1 μm. However, emulsions and microemulsions are not necessarily differentiated only by the globule size of the internal phase. Instead, they may also differ in one or more of the following defining properties:
(a) Microemulsions can form easily with little mixing energy needed and often without heating. They can form spontaneously, i.e. the ingredients in the correct proportions spontaneously form microemulsions once placed in a container. On the other hand, emulsions are thermodynamically unstable and require vigorous stirring with a high-shear mixer and usually need heating to a higher temperature, e.g. 75° C.
(b) The physical appearances of microemulsions and emulsions are different. Microemulsions are transparent (like water) because the globules in microemulsions are too small to refract light. Emulsions are usually white or cream in color.
(c) Microemulsions are thermodynamically stable at room temperature. Once the microemulsion is formed, it can be stable for many years in a sealed container and under normal storage condition. Emulsions have a tendency for the individual globules of the interior phase to coalesce (grow together) into larger and larger drops over time. Therefore, emulsions are generally stable for a relatively shorter period of time in bulk solution if it is left undisturbed, as the emulsion breaks or cracks to form completely separate phases, when compared to an otherwise identical microemulsion. It is believed that when an emulsion is adsorbed onto an adsorbent, however, it remains stable for a longer period of time than the same emulsion in bulk solution. Without being bound by theory, it is believed that upon adsorption, dispersion of the emulsion on the adsorbent retards coalescence of the interior phase globules with one another.
(d) The addition of specific proportions of each of the components and even their order of mixing can play a role in the formation of an emulsion, which also differentiates emulsions from microemulsions. Such knowledge and information on the formation of emulsions is understood by those skilled in the art (see, e.g., Physical Pharmacy by Alfred Martin, Lea and Febiger, 4th Ed. (1993).

In some embodiments, a drug-containing emulsion comprises emulsion globules having mean or modal diameters of greater than 100 nm (e.g., from about 120 nm to about 70 μm and from about 160 nm to about 10 μm). Typically, a microemulsion has globule diameters of less than about 100 nm (e.g., from about 30 to about 60 nm). In some embodiments, the drug containing emulsion of the solid dosage form composition is stable for at least one year when left in a closed container at 25° C. and can be an oil-in-water emulsion or a water-in-oil emulsion.

A drug-containing emulsion, drug-containing microemulsion, self-emulsifying oil composition, or self-microemulsifying oil composition can be adsorbed/absorbed onto adsorbents/absorbents (these two terms are collectively referred to as “adsorbent” or “adsorbents”). Adsorbents should be non-toxic and can include fine particles having diameters in the range of from about 25 nm to 300 μm (e.g., from about 50 nm to about 30 μm and from about 100 nm to about 20 μm). Suitable adsorbents include, but are not limited to, clays such as kaolin, bentonite, hectorite and colloidal magnesium aluminum silicate; silicon dioxide (CAB-O-SIL or AEROSIL®); magnesium trisilicate; aluminum hydroxide; magnesium hydroxide, magnesium oxide or talc. In some embodiments, the adsorbent can be silicon dioxide.

A dosage form, as described herein, can be in the form of a free-flowing, compressible powder, comprising: one or more of a drug-containing emulsion, drug-containing microemulsion, self-emulsifying oil composition, and self-microemulsifying oil composition; a pH modifier; and a solid particle adsorbent. In some embodiments, the drug-containing emulsion, drug-containing microemulsion, self-emulsifying oil composition, or self-microemulsifying oil composition, are adsorbed onto the solid particle adsorbent and form a free-flowing, compressible powder. Direct compression tableting excipients can also be added to the free-flowing compressible powder to improve its compressibility. Solid dosage forms can result from the tableting of the free-flowing powder using techniques understood by those of ordinary skill in the art.

The ratio of drug-containing emulsion, drug-containing microemulsion, self-emulsifying oil composition, or self-microemulsifying oil composition to solid particle can vary from about 1:20 to about 10:1 (e.g., from about 1:5 to about 2:1; from about 1:15 to about 3:1; from about 1:10 to about 1:1; from about 1:5 to about 5:1; from about 1:1 to about 8:1; from about 1:2 to about 4:1; and from about 1:8 to about 6:1).

A dosage form can be prepared by adsorbing the drug-containing emulsion, drug-containing microemulsion, self-emulsifying oil composition, or self-microemulsifying oil composition onto an adsorbent. In some embodiments, the adsorbent can be placed in a mixer and then the drug-containing emulsion, drug-containing microemulsion, self-emulsifying oil composition, and self-microemulsifying oil composition having a predetermined ratio to the adsorbent, can be poured into the mixer at constant stirring to achieve uniform adsorption of the emulsion to the adsorbent.

The resulting dosage form should be a free-flowing, compressible powder. In some embodiments, once the drug-containing emulsion, drug-containing microemulsion, self-emulsifying oil composition, or self-microemulsifying oil composition is adsorbed onto the solid particle the powder should resemble a dry powder (as far as observation with the eye can discern). In some embodiments, the powder can be free flowing as defined by the angle of repose test described below. Such characteristics can be more easily achieved with an o/w emulsion or microemulsion, partly due to the fact that the water in the external phase can partially evaporate during the incorporation process. There is an equilibrium amount of water that is retained on the solid particles. When adsorbing a w/o emulsion or microemulsion, there is a tendency for the powder to appear slightly “wet”. The proportion of emulsion or microemulsion to solid support is an important factor in determining the extent to which the powder remains free flowing and dry. However, with the proportions of solid support to emulsion or microemulsion as described above, it is possible to obtain a non-cohesive mixture. For the manufacture of a solid dosage form (e.g., compressed tablets), this mixture can then be combined with the other tableting components to obtain a compressible blend. In some embodiments, the compressible blend should be free flowing.

The extent to which a powder blend is free flowing is estimated by conducting an angle of repose test as detailed in a standard pharmaceutical text such as “The Theory and Practice of Industrial Pharmacy” by Lachman, Lieberman and Kanig (Lea and Febiger, publishers), hereby incorporated by reference. The static angle of repose test is preferred. When such a test is performed, the final powder blend can have an angle of repose less than about 42 degrees (e.g., less than about 40 degrees).

For a mixture of adsorbent and drug-containing emulsion, drug-containing microemulsion, self-emulsifying oil composition, or self-microemulsifying oil composition to form a free-flowing powder that can easily be compressed, the proportion of drug-containing emulsion, drug-containing microemulsion, self-emulsifying oil composition, or self-microemulsifying oil composition can be kept relatively low in the dosage form. Consequently, the drug-load in a drug-containing emulsion, drug-containing microemulsion, self-emulsifying oil composition, or self-microemulsifying oil composition can be a significant factor in formulating a stable, bioavailable and high drug-load composition.

It has been discovered that one can incorporate a wide range of drug loads into such free flowing, compressible powders while increasing the stability of the pharmaceutical emulsion or microemulsion. As provided herein, the emulsions and microemulsions can be formed from a wide range of weight ratios of water, oil, and emulsifying agent, and can be adsorbed onto a solid particulate adsorbent to form a free flowing, compressible powder.

A coating can also be applied to the individual particles of the dosage form powder, to agglomerates, granules or other larger particles incorporating multiple particles of the composition or to dosage forms or portions of dosage forms. The term “multiparticulate” as used herein means a composition in the form of multiple particles. Each particle may be an individual particle of the dosage form composition or may be an agglomerate, granule, or other larger particle incorporating or formed from multiple particles of the powder composition.

A final solid dosage form can contain from about 0.1 mg to about 1,000 mg of weakly ionizable drug per tablet (of e.g., 2.4 grams). In some embodiments, the solid dosage form contains from about 5 mg to about 500 mg (e.g., from about 10 mg to about 200 mg and from about 15 mg to about 100 mg) of weakly ionizable drug per tablet.

Solid dosage forms can further comprise excipients, such as, at least one of an effervescent agent and/or one disintegration agent; wherein the disintegration agent causes rapid dispersion and breaking up of the solid dosage form following oral administration.

This disclosure also provides for a method for preparing a solid dosage form, comprising the steps of: preparing a drug-containing emulsion, drug-containing microemulsion, self-emulsifying oil composition, or self-microemulsifying oil composition and converting the drug-containing emulsion, drug-containing microemulsion, self-emulsifying oil composition, or self-microemulsifying oil composition into a free-flowing, compressible powder by admixing the drug-containing emulsion, drug-containing microemulsion, self-emulsifying oil composition, or self-microemulsifying oil composition with a solid particle adsorbent. Solid dosage forms can then be prepared from the free-flowing compressible powder by the optional inclusion of excipients (fillers, disintegrants, etc.) followed by methods (e.g., tableting methods), known to those having ordinary skill in the art.

A drug-containing emulsion, drug-containing microemulsion, self-emulsifying oil composition, or self-microemulsifying oil composition can include, for example, from about 20% to about 40% (e.g., from about 20% to about 35%; from about 20% to about 32%; from about 25% to about 40%; from about 28% to about 40%; from about 25% to about 35%; and from about 28% to about 32%) by weight of a co-solvent; from about 4% to about 9% (from about 4% to about 8%; from about 4% to about 7%; from about 4.5% to about 9%; from about 5% to about 9%; from about 5% to about 8%; and from about 5% to about 7%) by weight of a pH modifier; from about 9% to about 18% (from about 9% to about 16%; from about 9% to about 14.5%; from about 9% to about 14%; from about 10% to about 18%; from about 12% to about 18%; from about 13% to about 18%; from about 10% to about 16%; and from about 12% to about 14%) by weight of purified water; from about 28% to about 56% (e.g., from about 28% to about 50%; from about 28% to about 46%; from about 32% to about 56%; from about 38% to about 56%; from about 30% to about 50%; from about 34% to about 47%; and from about 38% to about 44%) by weight of a surfactant; and from about 5% to about 10% (e.g., from about 5% to about 9%; from about 5% to about 8%; from about 6% to about 10%; from about 7% to about 10%; from about 6% to about 9%; and from about 7% to about 9%) by weight of a weakly ionizable drug. In some embodiments, the drug-containing emulsion, drug-containing microemulsion, self-emulsifying oil composition, or self-microemulsifying oil composition can include about 30% by weight of a co-solvent; about 6.67% by weight of a pH modifier; about 13.33% by weight of purified water; about 42% by weight of a surfactant; and about 8% by weight of a weakly ionizable drug.

In some embodiments, the formulation above can be combined with a solid particle adsorbent in a ratio from about 1:5 to about 5:1 (e.g., from about 1:5 to about 3:1; from about 1:5 to about 2:1; from about 1:3 to about 5:1; from about 1:2 to about 5:1; from about 1:3 to about 3:1; and from about 1:2 to about 2:1) by weight of drug-containing emulsion, drug-containing microemulsion, self-emulsifying oil composition, or self-microemulsifying oil composition to solid particle adsorbent.

In some embodiments, a dosage form, as described herein, can include from about 10% to about 20% (e.g., from about 10% to about 18%; from about 10% to about 16%; from about 12% to about 20%; from about 14% to about 20%; from about 12% to about 18%; and from about 14% to about 16%) by weight of a co-solvent; from about 2% to about 5% (from about 2% to about 4.5%; from about 2% to about 4%; from about 2.5% to about 5%; from about 3% to about 5%; from about 2.5% to about 4.5%; and from about 3% to about 4%) by weight of a pH modifier; from about 4% to about 9% (from about 4% to about 8%; from about 4% to about 7%; from about 4.5% to about 9%; from about 5% to about 9%; from about 5% to about 8%; and from about 5% to about 7%) by weight of purified water; from about 14% to about 28% (from about 14% to about 24%; from about 14% to about 22%; from about 16% to about 28%; from about 18% to about 28%; from about 16% to about 24%; and from about 18% to about 22%) by weight of a surfactant; from about 2% to about 6% (e.g., from about 2% to about 5%; from about 2% to about 4.5%; from about 3% to about 6%; from about 3.5% to about 6%; from about 2.75% to about 4.75%; from about 3% to about 4.5%; and from about 3.5% to about 4.5%) by weight of a weakly ionizable drug; and from about 35% to about 65% (e.g., from about 35% to about 60%; from about 35% to about 55%; from about 40% to about 65%; from about 45% to about 65%; from about 40% to about 60%; and from about 45% to about 55%) by weight of a solid particle adsorbent. In some embodiments, the formulation can include about 15% by weight of a co-solvent; about 3.33% by weight of a pH modifier; about 6.67% by weight of purified water; about 21% by weight of a surfactant; about 4% by weight of a weakly ionizable drug; and about 50% by weight of a solid particle adsorbent.

A co-solvent can be selected from the group consisting of Captex-355® (Glyceryl Tricaprylate/Caprate); Labrasol® (Caprylocaproyl Macrogol Glycerides); Labrafil M1944® (Oleoyl Macrogol Glycerides); Lauroglycol 90® (Propylene Glycol Monolaurate); Tween 80® (Polyoxyethylene (20) Sorbitan Monoleate); PEG 400 (Polyethylene Glycol); Capmul MCM® (Medium chain Mono- and Diglycerides); Capmul PG8® (Propylene Glycol Monocaprylate); Plurol Oleique® (polyglyceryl Oleate); Span 80® (Sorbitan Monoleate); Cremophor EL® (Polyoxyl 35 Castor Oil); Phosal 53MCT® (53% Phosphatidylcholine in Medium Chain Triglycerides); Corn oil; Oleic acid; Triethyl Citrate; Ethanol; Water; Acconon CC-6® (Polyoxyethylene 6 Caprylic/Capric Glycerides); PVP (Polyvinylpyrrolidone) K12; PVP K17; PVP K30; PVP K90; propylene glycol; and mixtures thereof. In some embodiments, the co-solvent is selected from Captex 355®; Capmul MCM®; PVP K12®; Propylene glycol; and mixtures thereof. In some embodiments, the co-solvent is about 3% Captex 355®; 5% Capmul MCM®; about 2% PVP K12®; and about 20% Propylene glycol.

A pH modifier can be any of those described previously or a mixture thereof.

A surfactant can be selected from the group consisting of Tween 85® (Polyoxyethylene (20) Sorbitan Trioleate); Tween 80® (Polyoxyethylene (20) Sorbitan Monoleate); Tween 20 (Polyoxyethylene (20) Sorbitan Monolaurate); Labrasol® (Caprylocaproyl Macrogol Glycerides); Cremophor EL® (Polyoxyl 35 Castor Oil); Triton X-100® (Octylphenol Ethoxylate); Oleic acid; Isopropyl Myristate; Ethyl Oleate; and mixtures thereof. In some embodiments, a surfactant can be Tween 80®.

A weakly ionizable drug can be any of those described previously or a mixture thereof.

A solid particle adsorbent can be any of those described previously or a mixture thereof.

Various ingredients and/or techniques can be used in combination with the dosage forms as described herein to enhance bioavailability, including for example, the inclusion of agents which aid in the site specific delivery of a drug-containing emulsion, drug-containing microemulsion, self-emulsifying oil composition, or self-microemulsifying oil composition, and agents which increase the rate of dissolution. The selected enhancement technique can be related to the route of drug absorption, i.e., paracellular or transcellular. Such techniques include, but are not limited to, the use of additional chemical penetration enhancers; mucoadhesive materials; effervescent agents including but not limited to effervescent couples; ion pairing or complexation agents; and the use of oil and/or surfactant drug carriers. Enhances in bioavailability can be achieved through structural and fluidity changes to the biological membranes induced by use of particular surfactants.

Further provided herein are dosage forms containing materials that aid release of the drug in a specific section of the gastrointestinal tract to promote site-specific delivery. The chosen site for drug release can be the most efficiently absorbing part of the gastrointestinal tract for the drug in question, or one that offers some other therapeutic advantage. The added materials can promote site-specific delivery by various mechanisms and the present disclosure is not limited to any one such mechanism. For example, the material may be metabolized by enzymes present in a specific part of the gastrointestinal tract, thus releasing the drug in that section. The materials used to promote site-specific absorption can be used as coatings and/or matrix materials and include, for example, sugars, polysaccharides, starches, polymers, and the like.

In some embodiments, a bioadhesive polymer can be included in the dosage form to increase the contact time between the dosage form and the mucosa of the most efficiently adsorbing section of the gastrointestinal tract. Non-limiting examples of bioadhesives include: Carbopol (various grades), sodium carboxy methylcellulose, methylcellulose, polycarbophil (Noveon AA-1), hydroxypropyl methylcellulose, hydroxypropyl cellulose, sodium alginate, and sodium hyaluronate.

Disintegration agents may also be employed, e.g., to aid in dispersion of the drug in the gastrointestinal tract.

In some embodiments, a disintegration agent can include any pharmaceutically acceptable effervescent agent or effervescent system. See, e.g., U.S. Pat. No. 5,178,878. In some embodiments, an effervescent agent or effervescent system can evolve gas by means of chemical reactions which take place upon exposure of the effervescent disintegration agent to water and/or to saliva in the mouth. The bubble or gas generating reaction is can, in some embodiments, be the result of the reaction of a soluble acid source and an alkali metal carbonate or carbonate source. The reaction of these two general classes of compounds produces carbon dioxide gas upon contact with water included in saliva.

The acid sources or acid may be any which are safe for human consumption and may generally include food acids, acid anhydrides and acid salts. Food acids include citric acid, tartaric acid, malic acid, fumaric acid, adipic acid, and succinic acids etc. Because these acids are directly ingested, their overall solubility in water is less important than it would be if the effervescent tablet formulations of the present invention were intended to be dissolved in a glass of water. Acid anhydrides and acid salts of the above described acids may also be used. Acid salts may include sodium, dihydrogen phosphate, disodium dihydrogen pyrophosphate, acid citrate salts and sodium acid sulfite.

Carbonate sources can include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate and amorphous calcium carbonate.

The effervescent disintegration agent(s) or system(s) of the present invention are not always based upon a reaction which forms carbon dioxide. Reactants which evolve oxygen or other gasses which are pediatrically safe can also be used. Where the effervescent agent includes two mutually reactive components, such as an acid source and a carbonate source, it is preferred that both components react completely. Therefore, an equivalent ratio of components which provides for equal equivalents is preferred. For example, if the acid used is diprotic, then either twice the amount of a mono-reactive carbonate base, or an equal amount of a di-reactive base should be used for complete neutralization to be realized. In some embodiments, however, the amount of either acid or carbonate source may exceed the amount of the other component. This may be useful to enhance taste and/or performance of a tablet containing an overage of either component. In this case, it is acceptable that the additional amount of either component may remain unreacted.

In some embodiments, a disintegration agent can be any suitable noneffervescent disintegration agent. Non-limiting examples of disintegration agents include: microcrystalline cellulose, croscarmelose sodium, crospovidone, starches and modified starches.

The various components described above may be present in layers within the dosage form or specialized shapes and geometric arrangements may be employed. In some embodiments, dosage forms as described herein can include drugs in addition to those carried in the drug-containing emulsion, drug-containing microemulsion, self-emulsifying oil composition, or self-microemulsifying oil composition.

Particles may be manufactured by granulation (wet or dry process), layering techniques, extrusion and spheronization or other pellet manufacturing methods. Dry granulation may be achieved through slugging or chilsonation of a powder mix (including adsorbed emulsion) that has the appearance of a dry powder. Layering may be done in a fluid bed apparatus or coating pan. The fine powder with the adsorbed emulsion (having the appearance of a dry powder) can be layered onto the starting material or cores. Aqueous or non-aqueous binders can be used to aid the adherence of the added material onto the cores. The choice of binder can be dictated, in part, by the nature of the emulsion or microemulsion, the weakly ionizable drug, and the pH modifier being utilized in a particular preparation. The binder should be tested for its effect on drug stability. Only the binders which do not negatively affect drug stability in the emulsion can be used. In some embodiments, layering can be done in a fluidized bed coater. In this apparatus, the bed of material can remain wet for a very short time and, hence, it can be possible to use a binder that may, at first sight, appear incompatible. In addition to the fine particle-adsorbed emulsion, other materials may be layered onto the starting material. These include, without limitation, the drug or additional amounts of the drug, the pH modifier, penetration enhancers, and other excipients. Non-limiting examples of the starting material or cores are nonpareils (sucrose) or microcrystalline cellulose seeds. The size of the multiparticulates is preferably up to about 3 mm. Coating of the dosage forms or the multiparticulates may be accomplished in a fluid bed coater or by other coating techniques. The multiparticulates may be packed into capsules.

Where a wet process, such as wet granulation or extrusion and spheronization is used, the emulsion or microemulsion can be used in a liquid phase or granulating fluid. In some embodiments, o/w emulsions or microemulsions can be used in a liquid phase in the wet process since this type of emulsion or microemulsion can be diluted with water to give the correct consistency for processing with the solid components and, furthermore, partial drying of the formed particulates can result in a product that has a dry appearance. Inclusion in the external aqueous phase of water-miscible, non-toxic, volatile organic solvents such as, for example, isopropyl alcohol or ethyl alcohol may be advantageous in facilitating the partial evaporation of the external phase of the emulsion or microemulsion from the formed particulates.

Also provided herein is a material which does not contain an enteric coat, but is retained in the oral cavity where the drug is released for absorption by the oral mucosa. When this material is utilized, the tablet may contain additional penetration enhancers, mucoadhesives or other agents to facilitate absorption in the oral cavity.

Tablets can be manufactured by wet granulation, dry granulation, direct compression, or any other tablet manufacturing technique. Orally disintegrating tablets may be relatively soft and can be made by direct compression in accordance with the disclosures in U.S. Pat. No. 5,178,878, which is hereby incorporated by reference herein. For peptides and other biological molecules, low compression forces are preferable because these substances are sensitive to compression forces. With such compounds, the conformation of the compound and the biological activity can change with the higher compression forces that are conventionally used in tablet manufacture.

In some embodiments, the tablet can be a layered tablet consisting of a layer of the active ingredients, as described above, within layers of diverse compositions. In some embodiments, the tablet can be a simple tablet of uniform composition. In some embodiments, a tablet can be up to about ¾ inch in size. Tablet hardness can be from about 1 Newton (“N”) and about 50 N (e.g., from about 15N to about 35N) for an uncoated tablet. Tablets that are intended to be coated, for example with an enteric coat, can be slightly harder, having hardness values ranging from about 20N to about 70N (e.g., from about 25N to about 50N). These values relate to the uncoated cores and, as expected, the hardness values of the tablets increase due to the addition of the coating layer.

In some embodiments, a tablet intended for vaginal administration can include fine particle powders as the filler and other excipients which reduce the potential for physical irritation or abrasion. In addition, the tablet can have a shape that facilitates insertion into the vagina. Non-limiting examples of such shapes include oval and diamond-shaped. The insertion of the tablet can be facilitated by the use of a special applicator device well-known in the industry for this purpose.

Tablets containing the solid dosage form can be coated with an enteric material. This can be done in a coating pan. Many of the modern, perforated pans have features which make for more efficient coating. As an example, the Hicoater (Vector Corporation, Iowa) may be used. In some embodiments, tablets within the pan are preheated and the pan can be rotated at a rate that allows gentle tumbling of the tablets. Many processes described above (such as rate of wetting of the material) made for the fluidized bed coater, can apply to the pan coater as well. The coating solution should be non-aqueous when effervescent material is incorporated within the preparation and the effervescence can be separated from the emulsion by a coating.

In some embodiments, precoating materials may also be used. Non-limiting examples of precoating materials include cellulose derivatives such as methylcellulose, hydroxypropyl cellulose, hydroxypropyl methylcellulose, materials sold under the trademark EUDRAGIT® (various grades which may be combined), and mixtures thereof.

In some embodiments, excipients, such as fillers, can be added to facilitate tableting. Non-limiting examples of fillers include: mannitol, dextrose, lactose, sucrose, and calcium carbonate. For a tablet intended to disintegrate in the oral cavity, the mass of the tablet should not exceed about 2.5 g. If an effervescent agent is included, the effervescence level in the tablet can be from about 5% to about 65% by weight based on the weight of the finished tablet.

The drug-containing emulsion, drug-containing microemulsion, self-emulsifying oil composition, or self-microemulsifying oil composition adsorbed onto a fine particle adsorbent may be incorporated into a suppository formed, for example, by molding. In some embodiments, the free-flowing powder can be mixed with a molten suppository base(s), poured into a mold, and allowed to set by cooling to ambient temperature. Suitable suppository bases include, but are not limited to, cocoa butter, polyethylene glycols, polyvinyl pyrrolidone, gelatin, gelatin/glycerin combinations, esterified fatty acids, polyoxyethylene sorbitans and polyoxyethylene sorbitan fatty acid esters. Various proprietary bases which may contain mixtures of different components are also available. Examples of proprietary bases are those sold under the trade names Imhausen, Witepsol and Gelucire. Various grades of each of these are available for specific applications. Mixtures of various bases may also be utilized to obtain a suppository with the required properties.

Various additives may be incorporated into the suppositories of the present invention including surfactants and absorption enhancers such as medium chain (C₈ to C₁₂) fatty acids and fatty acid esters including mono-, di-, and triesters of glycerol. Other shaping methods for forming the suppositories including cold molding and compression may also be used.

In some embodiments, the hydrophilic/hydrophobic nature of the suppository base can be different from the external phase of the emulsion, e.g., if an o/w emulsion or microemulsion is used, the suppository base should be a fatty (hydrophobic) base such as cocoa butter; and if a w/o emulsion or microemulsion is used, the suppository base should be hydrophilic such as, for example, a gelatin/glycerin base. These formulations can help to maintain the stability of the emulsion or microemulsion by preventing the formation of a miscible mixture between the external phase of the emulsion or microemulsion and the suppository base.

The dosage forms, as described herein, upon administration can achieve a shorter Tₘₐₓ of the weakly ionizable drug as compared to a composition of the weakly ionizable drug which is not as an emulsion, microemulsion, self-emulsifying oil composition, or self-microemulsifying oil composition, which does not comprise the pH modifier, and which is administered by the same route (e.g., oral or IV). In some embodiments, a weakly ionizable drug may demonstrate sufficient bioavailability when not formulated as a composition described herein, however, the use of the presently described dosage forms can result in a shorter Tₘₐₓ.

In some cases, the dosage forms as described herein, upon administration, may exhibit a Cₘₐₓ value that is from about 105% to about 195% (e.g., from about 105% to about 190%; from about 105% to about 180%; from about 105% to about 175%; from about 105% to about 170%; from about 105% to about 160%; from about 105% to about 155%; from about 105% to about 150%; from about 110% to about 195%; from about 120% to about 195%; from about 125% to about 195%; from about 130% to about 195%; from about 140% to about 195%; from about 145% to about 195%; from about 150% to about 195%; from about 115% to about 185%; from about 125% to about 175%; from about 130% to about 170%; from about 140% to about 160%; and from about 145% to about 155%) of the Cₘₐₓ exhibited by a composition comprising the weakly ionizable drug which is not an emulsion, microemulsion, self-emulsifying oil composition, or self-microemulsifying oil composition, which does not comprise the pH modifier, and which is administered by the same route.

In some cases, the dosage forms, upon administration, can achieve a Tₘₐₓ that is from about 25% to about 50% (e.g., from about 25% to about 45%; from about 25% to about 42%; from about 25% to about 40%; from about 25% to about 38%; from about 30% to about 50%; from about 33% to about 50%; from about 35% to about 50%; from about 38% to about 50%; from about 28% to about 47%; from about 32% to about 44%; and from about 35% to about 41%) of the Tₘₐₓ of the composition of the weakly ionizable drug which is not an emulsion, microemulsion, self-emulsifying oil composition, or self-microemulsifying oil composition, which does not comprise the pH modifier, and which is administered by the same route. In some embodiments, the dosage form can achieve a Tₘₐₓ that is from about 36% to about 40% of the Tₘₐₓ of a composition comprising the weakly ionizable drug that is not an emulsion, microemulsion, self-emulsifying oil composition, or self-microemulsifying oil composition, which does not comprise the pH modifier, and which is administered by the same route.

Similar percentage values for Cₘₐₓ and Tₘₐₓ are obtained for a composition of the weakly ionizable drug which is an emulsion, microemulsion, self-emulsifying oil composition, or self-microemulsifying oil composition, which does comprise the pH modifier, but which does not comprise a solid particle adsorbent when compared to that of the composition of the weakly ionizable drug which is not an emulsion, microemulsion, self-emulsifying oil composition, or self-microemulsifying oil composition, which does not comprise the pH modifier, and which is administered by the same route.

As illustrated in Example 7, the dosage forms, as described herein, can exhibit a Cₘₐₓ value that is similar to that achieved with a composition of the weakly ionizable drug which is an emulsion, microemulsion, self-emulsifying oil composition, or self-microemulsifying oil composition, which does comprise the pH modifier, but which does not comprise a solid particle adsorbent, and which is administered by the same route. In some embodiments, the Cₘₐₓ value is ±8% (e.g., ±6%; ±4%; and ±2%) of the Cₘₐₓ value of the dosage form lacking a solid particle adsorbent. Similarly, the dosage forms can exhibit a Tₘₐₓ value that is similar to that achieved with a composition of the weakly ionizable drug which is an emulsion, microemulsion, self-emulsifying oil composition, or self-microemulsifying oil composition, which does comprise the pH modifier, but which does not comprise a solid particle adsorbent, and which is administered by the same route. In some embodiments, the Tₘₐₓ value is ±20% (e.g., ±17%; ±15%; and ±12%) of the Tₘₐₓ value of the dosage form lacking a solid particle adsorbent.

In some embodiments, a weakly ionizable drug to be used in the presently described dosage forms can be poorly soluble, but exhibits acceptable absolute bioavailability. Typically, a poorly soluble drug can have a measured solubility ranging from slightly soluble to practically insoluble according to the table below. The presently described dosage forms can shorten the Tₘₐₓ of such drugs.

| | | | | | | Very | |
|---|---|---|---|---|---|---|---|
| | Very | Freely | | Sparingly | Slightly | Slightly | Practically |
| | Soluble | Soluble | Soluble | Soluble | Soluble | Soluble | Insoluble |
| Solubility | 1000 > S | 1000 > S > 100 | 100 > S > 33 | 33 > S > 10 | 10 > S > 1 | 1 > S > 0.1 | S < 0.1 |
| (S) range | | | | | | | |
| (mg/mL) | | | | | | | |

Absolute bioavailability can be determined by measuring the AUC achieved with a particular dosage form and route of administration as compared to that of an IV dosage form of a solubilized formulation of the drug. A poorly bioavailable drug has an absolute bioavailability that is less than about 30% (e.g., less than about 25%; less than about 20%; less than about 15%; less than about 10%; and less than about 5%) of the N dosage form of the drug. Drugs that display poor bioavailability can include drugs that are poorly adsorbed, and drugs that are degraded during passage through the gastrointestinal system, such as, for example, proteins, peptides and other substances of biological origin. On the other hand, a drug can have an acceptable bioavailability if it can measure an absolute bioavailability of greater than about 30% (e.g., greater than about 35%; greater than about 40%; greater than about 45%; greater than about 50%; greater than about 55%; greater than about 60%; greater than about 65%; greater than about 70%; greater than about 75%; greater than about 80%; greater than about 85%; greater than about 90%; and greater than about 95%) as compared to an N dosage form of the drug.

In some embodiments, the dosage forms, as described herein can contain weakly ionizable drugs which are poorly soluble but exhibit an acceptable bioavailability (e.g., greater than about 30% absolute bioavailability). In some embodiments, a dosage form of a drug can measure an absolute bioavailiabity of greater than about 85% as compared to an IV dosage form of the drug. In some embodiments, a dosage form of a drug can measure an absolute bioavailability of about 89% as compared to the N dosage form of the drug. Such formulations can also exhibit a shorter Tₘₐₓ than the drug which has not been formulated as described herein.

### Dosage Forms Comprising Meloxicam

In some embodiments, a dosage form can be prepared wherein the weakly ionizable drug can be meloxicam. Meloxicam is nonsteroidal anti-inflammatory drug (NSAID) having the following structure:

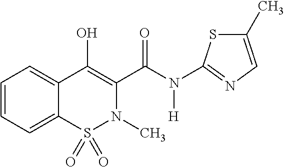

Meloxicam is also known as 4-hydroxy-2-methyl-N-(5-methyl-2-thiazolyl)-2H-1,2-benzothiazine-3-carboxamide-1,1-dioxide. Meloxicam is a weakly ionizable drug having pKa values of 1.1 and 4.2.

Meloxicam can be used in any of the dosage forms described above (i.e., as an emulsion, microemulsion, self-emulsifying oil composition, or self-microemulsifying oil composition), using any pH modifier and optional excipients as described previously.

A drug-containing emulsion, drug-containing microemulsion, self-emulsifying oil composition, or self-microemulsifying oil composition can include, for example, from about 20% to about 40% (e.g., from about 20% to about 35%; from about 20% to about 32%; from about 25% to about 40%; from about 28% to about 40%; from about 25% to about 35%; and from about 28% to about 32%) by weight of a co-solvent; from about 4% to about 9% (from about 4% to about 8%; from about 4% to about 7%; from about 4.5% to about 9%; from about 5% to about 9%; from about 5% to about 8%; and from about 5% to about 7%) by weight of a pH modifier; from about 9% to about 18% (from about 9% to about 16%; from about 9% to about 14.5%; from about 9% to about 14%; from about 10% to about 18%; from about 12% to about 18%; from about 13% to about 18%; from about 10% to about 16%; and from about 12% to about 14%) by weight of purified water; from about 28% to about 56% (e.g., from about 28% to about 50%; from about 28% to about 46%; from about 32% to about 56%; from about 38% to about 56%; from about 30% to about 50%; from about 34% to about 47%; and from about 38% to about 44%) by weight of a surfactant; and from about 5% to about 10% (e.g., from about 5% to about 9%; from about 5% to about 8%; from about 6% to about 10%; from about 7% to about 10%; from about 6% to about 9%; and from about 7% to about 9%) by weight of meloxicam. In some embodiments, the drug-containing emulsion, drug-containing microemulsion, self-emulsifying oil composition, or self-microemulsifying oil composition can include about 30% by weight of a co-solvent; about 6.67% by weight of a pH modifier; about 13.33% by weight of purified water; about 42% by weight of a surfactant; and about 8% by weight of meloxicam.

A drug-containing emulsion, drug-containing microemulsion, self-emulsifying oil composition, or self-microemulsifying oil composition can include about 3% by weight of Captex 355®; about 5% by weight Capmul MCM®; about 2% by weight PVP K12; about 20% by weight Propylene Glycol; about 20% by weight aqueous tris(hydroxymethyl)aminomethane; about 42% by weight Tween 80®; and about 8% by weight meloxicam.

In some embodiments, the formulation above can be combined with a solid particle adsorbent in a ratio from about 1:5 to about 5:1 (e.g., from about 1:5 to about 3:1; from about 1:5 to about 2:1; from about 1:3 to about 5:1; from about 1:2 to about 5:1; from about 1:3 to about 3:1; and from about 1:2 to about 2:1) by weight of drug-containing emulsion, drug-containing microemulsion, self-emulsifying oil composition, or self-microemulsifying oil composition to solid particle adsorbent. In some embodiments, the formulation above can be combined with a solid particle adsorbent in a ratio of 1:1 by weight of drug-containing emulsion, drug-containing microemulsion, self-emulsifying oil composition, or self-microemulsifying oil composition to solid particle adsorbent.

In some embodiments, a dosage form, as described herein, can include from about 10% to about 20% (e.g., from about 10% to about 18%; from about 10% to about 16%; from about 12% to about 20%; from about 14% to about 20%; from about 12% to about 18%; and from about 14% to about 16%) by weight of a co-solvent; from about 2% to about 5% (from about 2% to about 4.5%; from about 2% to about 4%; from about 2.5% to about 5%; from about 3% to about 5%; from about 2.5% to about 4.5%; and from about 3% to about 4%) by weight of a pH modifier; from about 4% to about 9% (from about 4% to about 8%; from about 4% to about 7%; from about 4.5% to about 9%; from about 5% to about 9%; from about 5% to about 8%; and from about 5% to about 7%) by weight of purified water; from about 14% to about 28% (from about 14% to about 24%; from about 14% to about 22%; from about 16% to about 28%; from about 18% to about 28%; from about 16% to about 24%; and from about 18% to about 22%) by weight of a surfactant; from about 2.5% to about 5% (e.g., from about 2.5% to about 4.5%; from about 2.5% to about 4%; from about 3% to about 5%; from about 3.5% to about 5%; from about 2.75% to about 4.75%; from about 3% to about 4.5%; and from about 3.5% to about 4.25%) by weight of meloxicam; and from about 35% to about 65% (e.g., from about 35% to about 60%; from about 35% to about 55%; from about 40% to about 65%; from about 45% to about 65%; from about 40% to about 60%; and from about 45% to about 55%) by weight of a solid particle adsorbent. In some embodiments, the formulation can include about 15% by weight of a co-solvent; about 3.33% by weight of a pH modifier; about 6.67% by weight of purified water; about 21.25% by weight of a surfactant; about 3.75% by weight of meloxicam; and about 50% by weight of a solid particle adsorbent. In some embodiments, the formulation can include about 1.5% by weight of Captex 355®; about 2.5% by weight Capmul MCM®; about 1% by weight PVP K12; about 10% by weight Propylene Glycol; about 10% by weight aqueous tris(hydroxymethyl)aminomethane; about 21% by weight Tween 80®; about 4% by weight meloxicam; and about 50% by weight silicon dioxide.

Dosage forms comprising meloxicam can, upon administration, achieve a shorter Tₘₐₓ compared to formulations of meloxicam that are not an emulsion, microemulsion, self-emulsifying oil composition, or self-microemulsifying oil composition, that do not comprise the pH modifier, and that are administered by the same route (see Example 5 and Busch, U. et al. Drug Metabolism and Disposition 26(6): 576-584 1998). In some embodiments, the Tₘₐₓ can range from about 94 to about 142 minutes (e.g., from about 94 to about 135 minutes; from about 94 to about 130 minutes; from about 94 to about 125 minutes; from about 94 to about 122 minutes; from about 94 to about 118 minutes; from about 98 to about 142 minutes; from about 105 to about 142 minutes; from about 110 to about 142 minutes; from about 114 to about 142 minutes; from about 118 to about 142 minutes; from about 100 to about 130 minutes; from about 108 to about 126 minutes; and from about 115 to about 121 minutes). In some embodiments, Tₘₐₓ ranges from about 116 to about 120 minutes. In some embodiments, a dosage form comprising meloxicam also comprises tris(hydroxymethyl)aminomethane and silicon dioxide (e.g., Zeopharm 5170).

Dosage forms comprising meloxicam can, upon administration, achieve a Cₘₐₓ can ranging from about 1.43 to about 2.14 μg/mL (e.g., from about 1.43 to about 2.10 μg/mL; from about 1.43 to about 2.06 μg/mL; from about 1.43 to about 1.98 μg/mL; from about 1.43 to about 1.92 μg/mL; from about 1.43 to about 1.86 μg/mL; from about 1.43 to about 1.80 μg/mL; from about 1.48 to about 2.14 μg/mL; from about 1.55 to about 2.14 μg/mL; from about 1.62 to about 2.14 μg/mL; from about 1.67 to about 2.14 μg/mL; from about 1.76 to about 2.14 μg/mL; from about 1.50 to about 2.10 μg/mL; from about 1.58 to about 1.98 μg/mL; from about 1.66 to about 1.89 μg/mL; and from about 1.72 to about 1.84 μg/mL). In some embodiments, Cₘₐₓ can range from about 1.75 to about 1.81 μg/mL.

In some cases, dosage forms comprising meloxicam can, upon administration, exhibit a Cₘₐₓ value that is from about 120% to about 180% (e.g., from about 120% to about 175%; from about 120% to about 166%; from about 120% to about 160%; from about 120% to about 156%; from about 125% to about 180%; from about 132% to about 180%; from about 140% to about 180%; from about 147% to about 180%; from about 125% to about 175%; from about 132% to about 168%; from about 138% to about 162%; and from about 145% to about 155%) of the Cₘₐₓ exhibited by a composition comprising meloxicam that is not an emulsion, microemulsion, self-emulsifying oil composition, or self-microemulsifying oil composition, that does not comprise the pH modifier, and that is administered by the same route. In such cases, the dosage forms, upon administration, can achieve a Tₘₐₓ that is from about 30% to about 46% (e.g., from about 30% to about 44%; from about 30% to about 42%; from about 30% to about 40%; from about 32% to about 46%; from about 34% to about 46%; from about 36% to about 46%; from about 32% to about 44%; and from about 34% to about 42%) of the Tₘₐₓ of a composition comprising meloxicam that is not an emulsion, microemulsion, self-emulsifying oil composition, or self-microemulsifying oil composition, that does not comprise the pH modifier, and that is administered by the same route. In some embodiments, the dosage form can achieve a Tₘₐₓ that is from about 36% to about 40% of the Tₘₐₓ of a composition comprising meloxicam that is not an emulsion, microemulsion, self-emulsifying oil composition, or self-microemulsifying oil composition, that does not comprise the pH modifier, and that is administered by the same route.

For example, in Example 4 the Cₘₐₓ for Mobic (i.e., a formulation of meloxicam that is not an emulsion, microemulsion, self-emulsifying oil composition, or self-microemulsifying oil composition and that does not comprise the pH modifier) exhibited a Cₘₐₓ of about 3.175 μg/mL while the dosage form comprising meloxicam, a pH modifier, and a solid particle adsorbent (i.e., a self-microemulsifying oil composition adsorbed onto a carrier) exhibited a Cₘₐₓ of 4.549 μg/mL in a study conducted in dogs. In addition, in that same study, the Tₘₐₓ of the Mobic formulation was 480 minutes, while the self-microemulsifying oil composition dosage form comprising meloxicam and pH modifier exhibited a Tₘₐₓ of 82.5 minutes.

In another example (see Example 7), the Cₘₐₓ for Mobic (i.e., a formulation of meloxicam that is not an emulsion, microemulsion, self-emulsifying oil composition, or self-microemulsifying oil composition and that does not comprise the pH modifier) exhibited a Cₘₐₓ of about 1.192 μg/mL while the dosage form comprising meloxicam, a pH modifier, and a solid particle adsorbent (i.e., a self-microemulsifying oil composition adsorbed onto a carrier) exhibited a Cₘₐₓ of 1.783 μg/mL in a study conducted in humans. Like the latter formulation, a dosage form comprising meloxicam and a pH modifier, but not having a solid particle adsorbent (i.e., a self-microemulsifying oil composition) exhibited a Cₘₐₓ of 1.803 μg/mL. In addition, in that same study, the Tₘₐₓ of the Mobic formulation was 308.4 minutes, while the self-microemulsifying oil composition adsorbed on a solid particle carrier comprising meloxicam and pH modifier exhibited a Tₘₐₓ of 118.2 minutes. As with Cₘₐₓ, the unadsorbed self-microemulsifying oil composition exhibited a similar Tₘₐₓ with a value of 105 minutes.

In some embodiments, dosage forms containing meloxicam as described herein can dissolve faster compared to a composition comprising meloxicam that is not an emulsion, microemulsion, self-emulsifying oil composition, or self-microemulsifying oil composition, that does not comprise the pH modifier, and that is administered by the same route. In some embodiments, from about 75 to about 100% (e.g., about 75%; about 80%; about 85%; about 90%; about 92%; about 94%; about 96%; and about 99%) of 15 mg of the dosage form dissolves in about 15 minutes in 900 mL DI water. In some embodiments, about 90% of 15 mg of the dosage form dissolves in about 15 minutes in 900 mL DI water. The dissolution rate of the dosage forms containing meloxicam as described herein can be compared to the dissolution rate of a composition comprising meloxicam that is not an emulsion, microemulsion, self-emulsifying oil composition, or self-microemulsifying oil composition, that does not comprise the pH modifier, and that is administered by the same route. In some embodiments, from about 40 to about 60% of 15 mg of the meloxicam dissolves in about 15 minutes in 900 mL DI water. In some embodiments, about 50% of 15 mg of the meloxicam dissolves in about 15 minutes in 900 mL DI water. The dissolution rates as described above are measured using an in-vitro dissolution assay, USP Dissolution, Apparatus 2.

Meloxicam exhibits anti-inflammatory, analgesic, and antifebrile activities. Meloxicam can be used, for example, in the treatment of inflammation and pain. In some embodiments, meloxicam can be used as a method of treating arthritis (e.g., osteoarthritis, rheumatoid arthritis, and juvenile rheumatoid arthritis), ankylosing spondylitis, fever, primary dysmenorrheal, pyrexia, asthma, bone resorption, cardiovascular diseases, nephrotoxicity, atherosclerosis, hypotension, lower back pain, and acute pain (e.g. treatment of post surgical pain, treatment of pain resulting from battle field wounds, and migraine headaches). Meloxicam may be especially effective for treatment of all types of pain associated with inflammation.

Various publications are cited throughout this application. These publications are hereby incorporated by reference.

### EXAMPLES

### Example 1

### Formulation of a Meloxicam Self-Emulsifying Oil Composition

A meloxicam self-emulsifying oil composition was prepared using the following components shown as percent by weight values:

| | | Amount |
|---|---|---|
| Component | Supplier | (weight percent) |
| Captex 355 | Abitec Corp (Janesville, WI) | 3% |
| Capmul MCM | Abitec Corp (Janesville, WI) | 5% |
| PVP K12 | | 2% |
| Propylene glycol | EM Sciences (Gibbstown, NJ) | 20% |
| Trizma ® buffer | Sigma Aldrich (MO, USA) | 20% |
| Tween 80 | Uniqema (New Castle, DE) | 42.5% |
| Meloxicam | | 7.5% |

Trizma® buffer was prepared by mixing 7.5 g of tris(hydroxymethyl)aminomethane base with 15 g of water. The remaining components, with the exception of meloxicam, were blended in a glass beaker with the aid of a magnetic stirrer in the order shown in the formula above. The beaker was placed in a 60° C. water bath and the components were blended for approximately 30 minutes until a transparent solution was obtained. Meloxicam (7.5%) was then added to the blend. The beaker was covered with a paraffin film and kept in the 60° C. water bath, and the solution was mixed for an additional hour until a transparent yellow solution was obtained. If the preparation turned hazy, orange, or dark yellow, it would indicate that the mixing method had failed to solubilize the meloxicam and the blend was discarded.

### Example 2

### Adsorption of Meloxicam Self-Emulsifying Oil Composition on Silica and Silicates

The meloxicam self-emulsifying oil composition of Example 1 was added in small aliquots and adsorbed under gentle mixing on three different types of powders: silicon dioxide 3.6 μm (GL100), silicon dioxide 300 μM (ZeoPharm 5170), and MgAl silicate 80 μm (Neusilin US2) each at a 1:1 wt/wt ratio. All three powder blends were stored in tightly closed glass containers until used.

### Example 3

### In Vitro Dissolution Studies

In vitro dissolution studies were performed on the meloxicam self-emulsifying oil composition adsorbed on both types of silicon dioxide and the MgAl silicate from Example 3. Dissolution was performed in 900 mL of DI water. Dissolution studies were conducted in a USP type 2 apparatus (Varian Inc., Cary, N.C.). Paddle speed and bath temperature were set to 50 rpm and 37° C., respectively. Samples (5 mL) were withdrawn at 5, 10, 15, 30, 45, and 60 minutes and analyzed by UV spectroscopic methods at 360 nm (Varian Cary, Australia). Dissolution experiment was performed in duplicates unless otherwise specified.

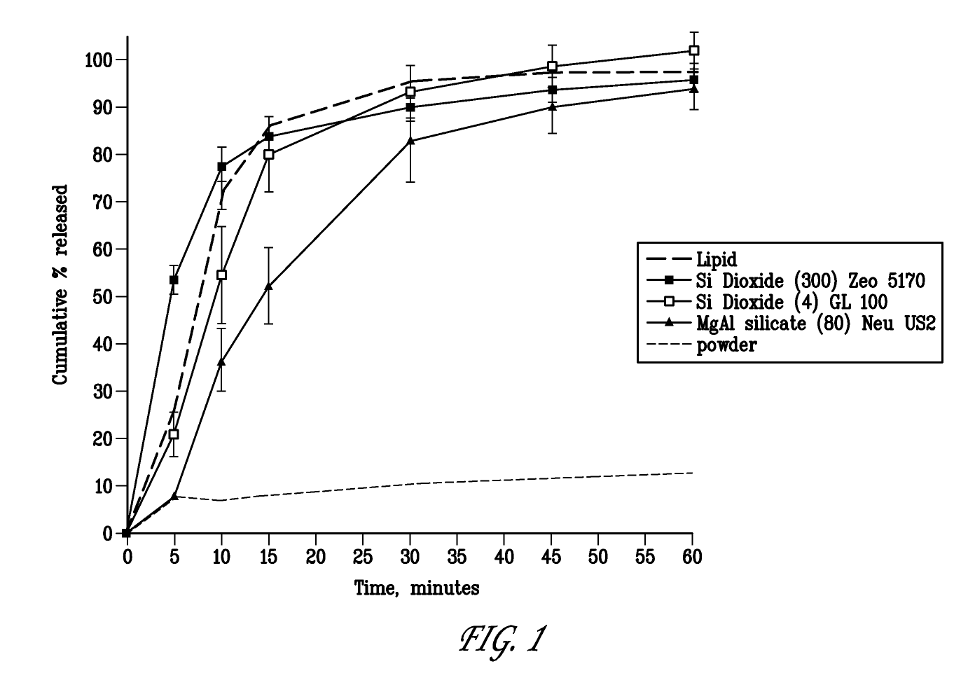

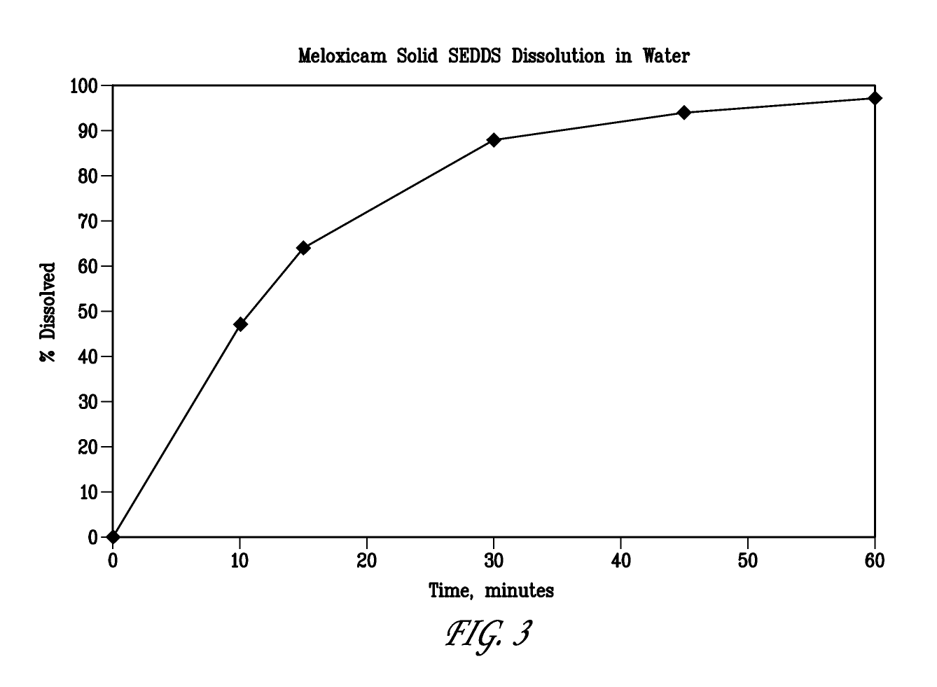

FIG. 1 shows the dissolution profiles in DI water of meloxicam (plain drug in powder form), the unadsorbed self-emulsifying oil composition, and the self-emulsifying oil composition adsorbed on both types of silicon dioxide and MgAl silicate as described in Example 2. Each formulation contained 15 mg of meloxicam. Only 12% of meloxicam powder dissolved in water when introduced to the dissolution medium. In contrast, >96% of meloxicam dissolved after 60 minutes when introduced as a self-emulsifying oil formulation (see FIG. 3). Similarly, >90% of the meloxicam dissolved after 60 minutes when introduced as the meloxicam self-emulsifying oil composition adsorbed on the silicon dioxides and the MgAl silicate. Both silicon dioxide adsorbents showed similar release profiles as the oil formulation alone, whereas the release of meloxicam from the MgAl silicate formulation was slower at the initial time points.

### Example 4

### In Vivo Dog Study

A formulation of a meloxicam self-microemulsifying oil composition adsorbed to solid particles was evaluated in an awake dog model in a nonrandom protocol design in the same dog group. Two forms of the meloxicam self-microemulsifying oil composition formulation were used as test articles in the study. One formulation consisted of hard gelatin capsules loaded with a self-microemulsifying oil composition comprising tris(hydroxy)aminomethane and meloxicam adsorbed onto a carrier, Zeopharm 5170, and the other consisted of hard gel capsules containing only the self-microemulsifying oil composition of meloxicam and tris(hydroxy)aminomethane. Both formulations were administered orally. Two commercial formulations containing meloxicam were also evaluated, Mobic® tablets administered orally and Metacam® for injection administered intravenously. Meloxicam dose was 7.5 or 8.1 mg for the orally administered and 2 mg for the intravenously administered test articles.

For each formulation 4 or 5 dogs were studied. Prior to administration, the animals were fasted overnight and approximately 2 hours post-dose, the animals had access to regular rations for the remaining portion of the study. The animals were dosed multiple times over the course of the study with at least a two-week recovery period between dosing phases. Each formulation was administered as a single dose, with the intravenous dosages occurring in the cephalic vein.

Whole blood and serum samples were collected at various time points over the course of 480 to 1440 minutes post-dose depending on the route of administration. Blood samples were collected from the cephalic or saphenous veins. The samples were stored at room temperature for about 1 hour or until being processed for serum. The samples were centrifuged at 3200 RPM for 10 minutes at 22-26° C. Serum samples were stored at 4±5° C. until analyzed.

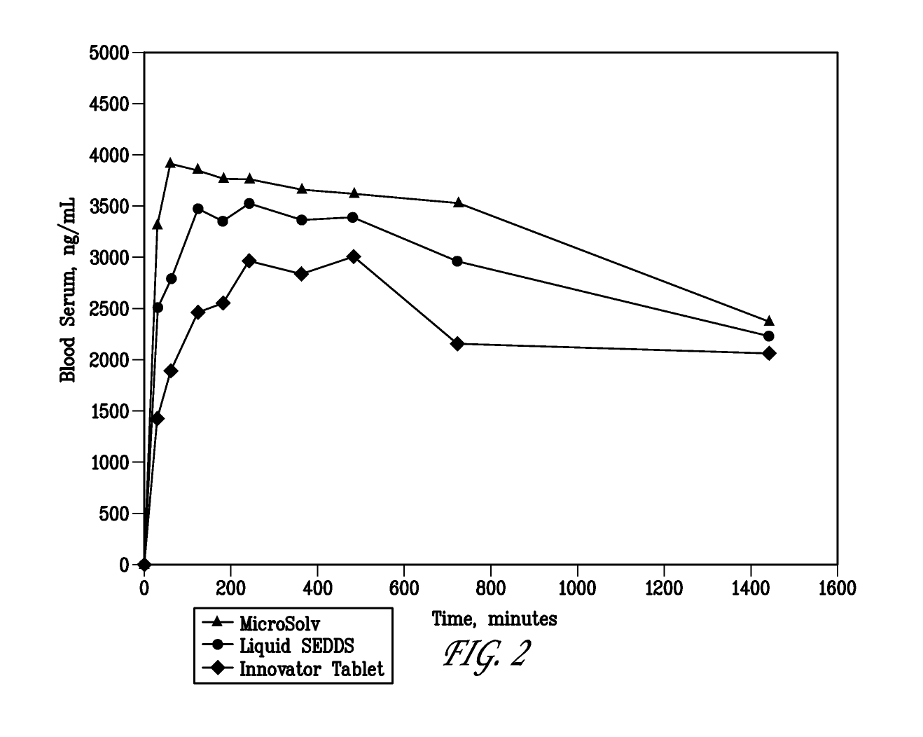

The samples were evaluated using LC/MS/MS. Table 1 and FIG. 2 detail the results of the calculated pharmacokinetic parameters obtained in this study.

TABLE 1

| | | Tₘₐₓ (min, max), | Cₘₐₓ, ng/mL | AUC_{inf}, min * ng/mL |
|---|---|---|---|---|
| | Dose, mg | minutes | (±SD) | (±SD) |
| Metacam IV | 2 | 2 (2, 2) | 1463 ± 276 | 2.25 × 10⁶ ± 5.31 × 10⁴ |
| Mobic (tablet) | 7.5 | 480 (240, 720) | 3175 ± 566 | 1.14 × 10⁷ ± 5.68 × 10⁶ |
| self- | 8.1 | 82.5 (30, 120) | 4549 ± 1419 | 8.98 × 10⁶ ± 2.32 × 10⁶ |
| microemulsifying | | | | |
| oil composition | | | | |
| adsorbed onto a | | | | |
| carrier | | | | |
| self- | 7.5 | 277.5 (30, 720) | 3792 ± 372 | 9.72 × 10⁶ ± 4.55 × 10⁶ |
| microemulsifying | | | | |
| oil composition | | | | |

The pharmacokinetic profiles indicate a slow elimination phase for meloxicam, as known from the literature. However, the meloxicam orally administered self-microemulsifying oil composition formulations show faster absorption in both the pharmacokinetics profiles and in the calculated pharmacokinetic parameters than achieved with orally administered Mobic.

### Example 5

### Formulation of a Meloxicam Self-Emulsifying Oil Composition

A meloxicam self-emulsifying oil composition was prepared using the following components shown as percent by weight values:

| | | Amount |
|---|---|---|
| Component | Supplier | (weight percent) |
| Captex 355 | Abitec Corp | 3% |
| | (Janesville, WI) | |
| Capmul MCM | Abitec Corp | 5% |
| PVP K12 | (Janesville, WI) | 2% |
| Propylene glycol | EM Sciences | 20% |
| | (Gibbstown, NJ) | |
| Tris(hydroxymethyl)aminomethane | Sigma Aldrich | 6.67% |
| Purified Water | (MO, USA) | 13.33% |
| Tween 80 | Uniqema | 42% |
| Meloxicam | (New Castle, DE) | 8% |

The components, with the exception of meloxicam, were blended in a glass beaker with the aid of a magnetic stirrer in the order shown in the formula above. The beaker was placed in a 60° C. water bath and the components were blended for approximately 30 minutes until a transparent solution was obtained. Meloxicam (8%) was then added to the blend. The beaker was covered with a paraffin film and kept in the 60° C. water bath, and the solution was mixed for an additional hour until a transparent yellow solution was obtained. If the preparation turned hazy, orange, or dark yellow, it would indicate that the mixing method had failed to solubilize the meloxicam and the blend was discarded.

### Example 6

### Adsorption of Meloxicam Self-Emulsifying Oil Composition on Silica and Silicates

The meloxicam self-emulsifying oil composition of Example 5 was added in small aliquots and adsorbed under gentle mixing onto Zeopharm 5170 (Silicon Dioxide (Precipitated Amorphous Silica)) at a 1:1 wt/wt ratio. The powder blend was stored in a tightly closed glass container until used.

### Example 7

### In Vivo Human Study

A formulation of a meloxicam self-microemulsifying oil composition adsorbed to solid particles was evaluated in a partially randomized, open-label study of certain pharmacokinetic parameters in normal healthy volunteers. Two forms of the meloxicam self-microemulsifying oil composition formulation were used as test articles in the study. One formulation (formulation A) consisted of hard gelatin capsules loaded with a self-microemulsifying oil composition comprising tris(hydroxy)aminomethane and meloxicam adsorbed onto a carrier, Zeopharm 5170 (see Example 6), and the other (formulation B) consisted of a liquid formulation containing only the self-microemulsifying oil composition of meloxicam and tris(hydroxy)aminomethane (see Example 5). A commercial tablet formulation (formulation C), Mobic®, containing meloxicam was also evaluated. Meloxicam dose was 15 mg regardless of formulation, and all formulations were administered orally.

The study consisted of a total of eighteen healthy adult volunteers (male and female). Testing was conducted over three phases, with a minimum fourteen day washout period between each phase of the study. Test formulation A and C were randomly dosed during phases 1 and 2, while test formulation B was dosed entirely during phase 3. Prior to administration, subjects fasted at least 10.5 hours and at least 24 hours after each phase of dosing. Each formulation was administered as a single dose, with approximately 240 mL of room temperature water.

Twenty-seven blood samples per subject per phase were collected at various time points over the course of 96 hours following dosing. Pharmacokinetic sampling occurred within 90 minutes prior to dosing (0 hour) and after dose administration at 0.5, 1, 1.5, 2, 2.5, 3, 3.5, 4, 4.5, 5, 5.5, 6, 6.5, 7, 7.5, 8, 8.5, 9, 9.5, 10, 12, 16, 24, 48, 72, and 96 hours. The samples were stored at room temperature for about 1 hour or until being processed for serum. The samples were centrifuged at 3000 RPM for 10 minutes at 4° C. (±10° C.). Serum samples were stored at −20±10° C. until analyzed. The time between sample collection and freezer storage did not exceed 1.5 hours.

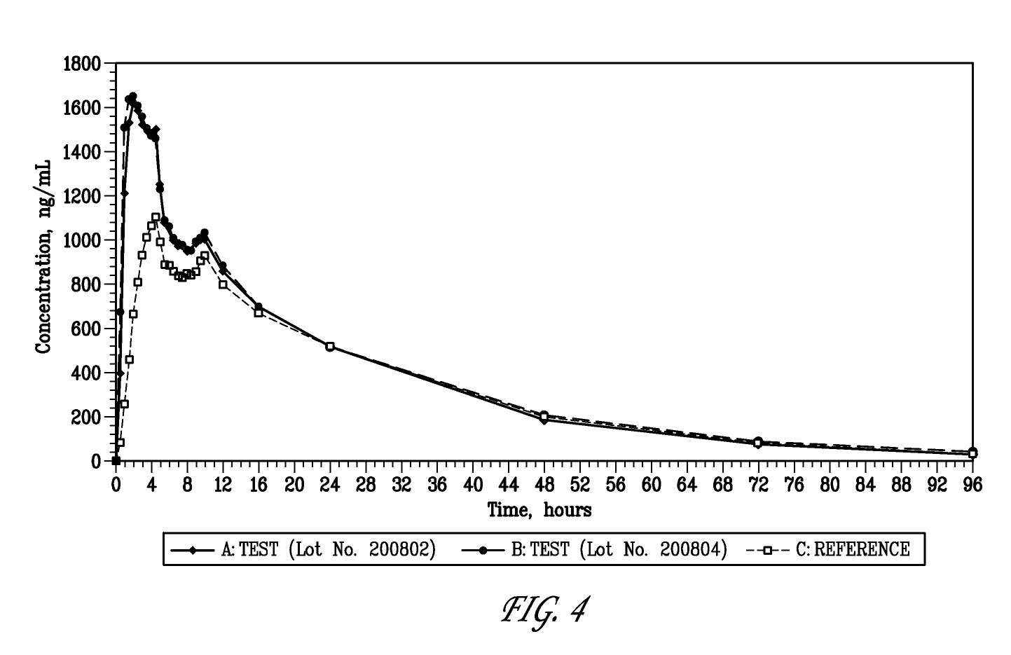

The samples were evaluated using a validated bioanalytical method and according to the Bioanalytical Laboratory's Standard Operating Procedures and FDA guidelines. Table 2 and FIG. 4 detail the results of the calculated pharmacokinetic parameters obtained in this study.

TABLE 2

| | | | AUC_{inf}, |
|---|---|---|---|
| | Tₘₐₓ minutes | Cₘₐₓ, ng/mL | hr * ng/mL |
| Formulation | (mean) | (mean) | (mean) |
| C—Mobic | 308.4 | 1191.88 | 31532.13 |
| (tablet) | | | |
| A—self- | 118.2 | 1783.33 | 35072.99 |
| microemulsifying | | | |
| oil composition | | | |
| adsorbed onto a | | | |
| carrier | | | |
| B—self- | 105 | 1803.39 | 36994.07 |
| microemulsifying | | | |
| oil composition | | | |

As in Example 4, the pharmacokinetic profiles indicate a slow elimination phase for meloxicam. However, formulations A and B show faster absorption in both the pharmacokinetics profiles and in the calculated pharmacokinetic parameters than achieved with commercially available formulation C.

### Example 8

### In Vitro Dissolution Studies

In vitro dissolution studies were performed on the meloxicam self-emulsifying oil composition adsorbed on silicon dioxide and the tablet formulation of meloxicam (Mobic). Dissolution was performed in 900 mL of DI water, aqueous solutions having pH values of pH 6.8 and pH 4.5, and a solution of 0.1 N HCl. Dissolution studies were conducted in a USP type 2 apparatus (Varian Inc., Cary, N.C.). Paddle speed and bath temperature were set to 50 rpm and 37° C., respectively. Samples (5 mL) were withdrawn at 5, 10, 15, 30, 45, and 60 minutes and analyzed by UV spectroscopic methods at 360 nm (Varian Cary, Australia). Dissolution experiment was performed in duplicates unless otherwise specified.

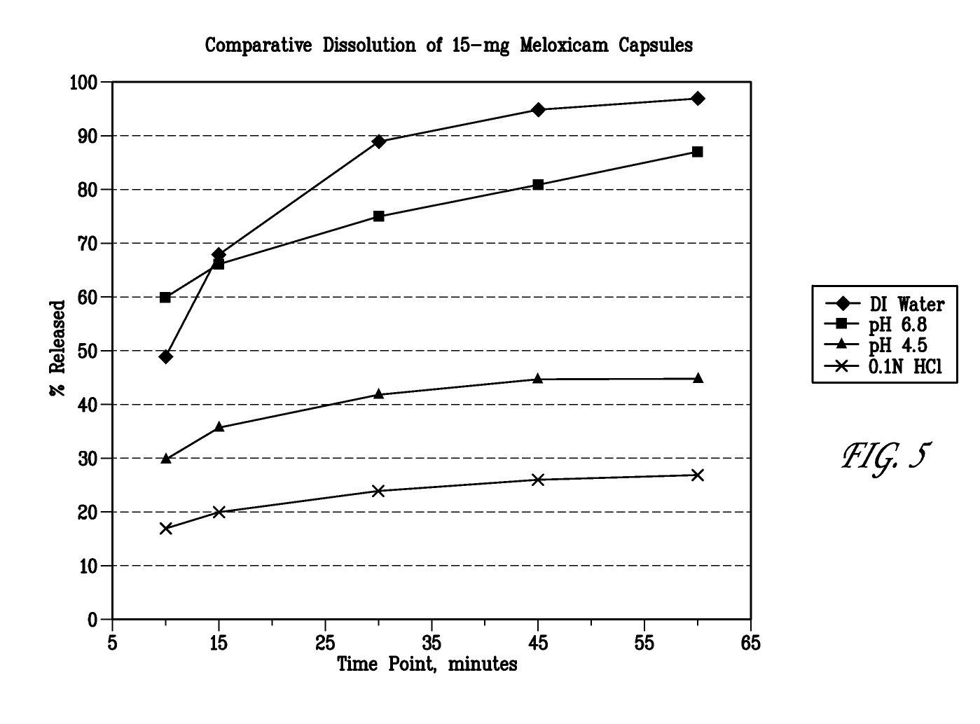

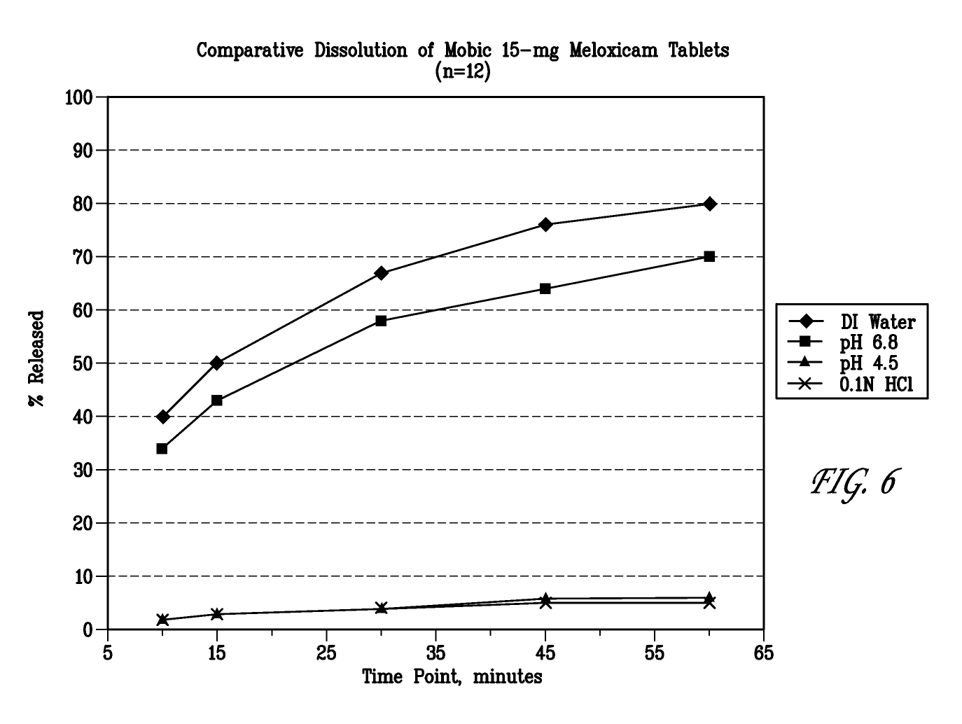

As shown in FIGS. 5 and 6, the meloxicam self-emulsifying oil composition adsorbed on silicon dioxide formulation exhibited a superior dissolution profile when compared to the tablet formulation.

A number of embodiments of the invention have been described. Nevertheless, it will be understood that various modifications may be made without departing from the spirit and scope of the invention. Accordingly, other embodiments are within the scope of the following claims.

What is claimed is:

1. A dosage form comprising:
a) a drug-containing emulsion having globules of diameter greater than 100 nm, wherein the drug-containing emulsion comprises a weakly ionizable drug and a pH modifier, wherein the pH modifier increases the solubility of the weakly ionizable drug in an aqueous phase as compared to the solubility of the weakly ionizable drug in the absence of the pH modifier, and wherein the weakly ionizable drug has a pKa of from about 1 to about 14; and
b) a solid particle adsorbent,
wherein the drug-containing emulsion is adsorbed to the solid particle adsorbent to form a free-flowing compressible powder.

2. The dosage form of claim 1, wherein the emulsion globules have diameters of from about 120 nm to about 70 μm.

3. The dosage form of claim 1, wherein the drug-containing emulsion comprises from about 1% to about 50% by weight of the weakly ionizable drug based on the total weight of both phases of the emulsion.

4. The dosage form of claim 1, wherein the drug-containing emulsion is an oil-in-water emulsion.

5. The dosage form of claim 1, wherein the drug-containing emulsion is a water-in-oil emulsion.

6. The dosage form of claim 1, wherein the solid particle is selected from the group consisting of kaolin; bentonite; hectorite; colloidal magnesium aluminum silicate; silicon dioxide; magnesium trisilicate; aluminum hydroxide; magnesium hydroxide; magnesium oxide; and talc.

7. The dosage form of claim 1, wherein the weakly ionizable drug is selected from the group consisting of meloxicam; atropine; chloramphenicol; chlorothiazide; chlorpromazine; cimetidine; diazepam; diltiazem; diphenhydramine; disopyramide; flufenamic acid; furosemide; haloperidol; imipramine; lidocaine; phenobarbital; phenyloin; procainamide; propafenone; propranolol; tetracaine; trimethoprim; and verapamil.

8. The dosage form of claim 7, wherein the weakly ionizable drug is meloxicam.

9. The dosage form of claim 1, wherein the pH modifier is selected from the group consisting of: sodium carbonate; potassium carbonate; magnesium carbonate; sodium bicarbonate; potassium bicarbonate; disodium hydrogen phosphate; sodium dihydrogen phosphate; dipotassium hydrogen phosphate; potassium dihydrogen phosphate; tris(hydroxymethyl)aminomethane; citric acid; tartaric acid; amalic acid; fumeric acid; adipic acid; and succinic acid.

10. The dosage form of claim 9, wherein the pH modifier is tris(hydroxymethyl)aminomethane.

11. A dosage form comprising:
a. a drug-containing microemulsion having globules of diameter less than 100 nm, wherein the drug-containing microemulsion comprises a weakly ionizable drug and a pH modifier, wherein the pH modifier increases the solubility of the weakly ionizable drug in an aqueous phase as compared to the solubility of the weakly ionizable drug in the absence of the pH modifier, and wherein the weakly ionizable drug has a pKa of from about 1 to about 14; and
b. a solid particle adsorbent,
wherein the drug-containing microemulsion is adsorbed to the solid particle adsorbent to form a free-flowing compressible powder.

12. The dosage form of claim 11, wherein the drug-containing microemulsion comprises from about 1% to about 50% by weight of the weakly ionizable drug based on the total weight of both phases of the microemulsion.

13. The dosage form of claim 11, wherein the drug-containing microemulsion is an oil-in-water emulsion.

14. The dosage form of claim 11, wherein the drug-containing microemulsion is a water-in-oil emulsion.

15. The dosage form of claim 11, wherein the solid particle is selected from the group consisting of kaolin; bentonite; hectorite; colloidal magnesium aluminum silicate; silicon dioxide; magnesium trisilicate; aluminum hydroxide; magnesium hydroxide; magnesium oxide; and talc.

16. The dosage form of claim 11, wherein the weakly ionizable drug is selected from the group consisting of meloxicam; atropine; chloramphenicol; chlorothiazide; chlorpromazine; cimetidine; diazepam; diltiazem; diphenhydramine; disopyramide; flufenamic acid; furosemide; haloperidol; imipramine; lidocaine; phenobarbital; phenyloin; procainamide; propafenone; propranolol; tetracaine; trimethoprim; and verapamil.

17. The dosage form of claim 16, wherein the weakly ionizable drug is meloxicam.

18. The dosage form of claim 11, wherein the pH modifier is selected from the group consisting of: sodium carbonate; potassium carbonate; magnesium carbonate; sodium bicarbonate; potassium bicarbonate; disodium hydrogen phosphate; sodium dihydrogen phosphate; dipotassium hydrogen phosphate; potassium dihydrogen phosphate; tris(hydroxymethyl)aminomethane; citric acid; tartaric acid; amalic acid; fumeric acid; adipic acid; and succinic acid.

19. The dosage form of claim 18, wherein the pH modifier is tris(hydroxymethyl)aminomethane.

20. A dosage form comprising:
a. a drug-containing emulsion having globules of diameter greater than 100 nm, wherein the drug-containing emulsion comprises meloxicam and a pH modifier, and wherein the pH modifier increases the solubility of meloxicam in an aqueous phase as compared to the solubility of meloxicam in the absence of the pH modifier; and
b. a solid particle adsorbent,
wherein the drug-containing emulsion is adsorbed to the solid particle adsorbent to form a free-flowing compressible powder.

21. The dosage form of claim 20, wherein the dosage form upon administration achieves a shorter Tₘₐₓ compared to a composition comprising meloxicam that is not an emulsion, that does not comprise the pH modifier, and that is administered by the same route.

22. The dosage form of claim 21, wherein the Tₘₐₓ ranges from about 94 to about 142 minutes.

23. The dosage form of claim 22, wherein the Tₘₐₓ ranges from about 116 to about 120 minutes.

24. The dosage form of claim 23, wherein the dosage form dissolves faster in water compared to a composition comprising meloxicam that is not an emulsion, that does not comprise the pH modifier, and that is administered by the same route.

25. The dosage form of claim 24, wherein from about 75 to about 100% of 15 mg of the dosage form dissolves in about 15 minutes in 900 mL water.

26. The dosage form of claim 25, wherein about 90% of 15 mg of the dosage form dissolves in about 15 minutes in 900 mL water.

27. A dosage form comprising:
a. a drug-containing microemulsion having globules of diameter less than 100 nm, wherein the drug-containing microemulsion comprises meloxicam and a pH modifier, and wherein the pH modifier increases the solubility of meloxicam in an aqueous phase as compared to the solubility of meloxicam in the absence of the pH modifier; and
b. a solid particle adsorbent,
wherein the drug-containing microemulsion is adsorbed to the solid particle adsorbent to form a free-flowing compressible powder.

28. The dosage form of claim 27, wherein the dosage form upon administration achieves a shorter Tₘₐₓ compared to a composition comprising meloxicam that is not a microemulsion, that does not comprise the pH modifier, and that is administered by the same route.

29. The dosage form of claim 28, wherein the Tₘₐₓ ranges from about 94 to about 142 minutes.

30. The dosage form of claim 29, wherein the Tₘₐₓ ranges from about 116 to about 120 minutes.

31. The dosage form of claim 27, wherein the dosage form dissolves faster in water compared to a composition comprising meloxicam that is not a microemulsion, that does not comprise the pH modifier, and that is administered by the same route.

32. The dosage form of claim 31, wherein from about 75 to about 100% of 15 mg of the dosage form dissolves in about 15 minutes in 900 mL water.

33. The dosage form of claim 32, wherein about 90% of 15 mg of the dosage form dissolves in about 15 minutes in 900 mL water.

34. A self-emulsifying oil composition comprising one or more oils, a weakly ionizable drug and a pH modifier, wherein the self-emulsifying oil composition forms a drug-containing emulsion having globules of diameter greater than 100 nm when exposed to an aqueous phase, wherein the pH modifier increases the solubility of the weakly ionizable drug in an aqueous phase as compared to the solubility of the weakly ionizable drug in the absence of the pH modifier, and wherein the weakly ionizable drug has a pKa of from about 1 to about 14.

35. A self-microemulsifying oil composition comprising one or more oils, a weakly ionizable drug and a pH modifier, wherein the self-microemulsifying oil composition forms a drug-containing microemulsion having globules of diameter less than 100 nm when exposed to an aqueous phase, wherein the pH modifier increases the solubility of the weakly ionizable drug in an aqueous phase as compared to the solubility of the weakly ionizable drug in the absence of the pH modifier, and wherein the weakly ionizable drug has a pKa of from about 1 to about 14.

36. A method for preparing a dosage form of a drug-containing emulsion comprising the steps of:
(a) preparing a drug-containing emulsion comprising a weakly ionizable drug, wherein the weakly ionizable drug has a pKa ranging from about 1 to about 14, and a pH modifier;
(b) converting the drug-containing emulsion into a free-flowing compressible powder by admixing the drug-containing emulsion with a solid particle adsorbent.

37. A method for preparing a dosage form of a drug-containing microemulsion, comprising the steps of:
(a) preparing a drug-containing microemulsion comprising a weakly ionizable drug, wherein the weakly ionizable drug has a pKa ranging from about 1 to about 14, and a pH modifier;
(b) converting the drug-containing microemulsion into a free-flowing compressible powder by admixing the drug-containing microemulsion with a solid particle adsorbent.

38. A method for preparing a dosage form of a drug-containing self-emulsifying oil composition, comprising the steps of:
(a) preparing a drug-containing self-emulsifying oil composition comprising one or more oils, a weakly ionizable drug, wherein the weakly ionizable drug has a pKa ranging from about 1 to about 14, and a pH modifier;
(b) converting the drug-containing self-emulsifying oil composition into a free-flowing compressible powder by admixing the drug-containing self-emulsifying drug delivery system with a solid particle adsorbent.

39. A method for preparing a powder form of a drug-containing self-microemulsifying oil composition, comprising the steps of:
(a) preparing a drug-containing self-microemulsifying oil composition comprising one or more oils, a weakly ionizable drug wherein the weakly ionizable drug has a pKa ranging from about 1 to about 14, and a pH modifier;
(b) converting the drug-containing self-microemulsifying oil composition into a free-flowing compressible powder by admixing the drug-containing self-microemulsifying oil composition with a solid particle adsorbent.

40. A method of administering a drug to a mammal comprising the steps of:
(a) preparing a drug-containing emulsion comprising a weakly ionizable drug, wherein the weakly ionizable drug has a pKa ranging from about 1 to about 14, and a pH modifier;
(b) converting the drug-containing emulsion into a free-flowing compressible power by admixing the drug-containing emulsion with a solid particle adsorbent; and
(c) orally administering the free-flowing compressible powder to the mammal.

41. A method of treating pain in a mammal, the method comprising administering to the mammal a dosage form from any one of claims 20 and 27.

42. A method of treating arthritis in a mammal, the method comprising administering to the mammal a dosage form from any one of claims 20 and 27.

43. A method of treating ankylosing spondylitis in a mammal, the method comprising administering to the mammal a dosage form from any one of claims 20 and 27.

44. A method of treating fever, the method comprising administering to the mammal a dosage form from any one of claims 20 and 27.

45. A method of treating asthma in a mammal, the method comprising administering to the mammal a dosage form from any one of claims 20 and 27.

46. A method of treating cardiovascular disease in a mammal, the method comprising administering to the mammal a dosage form from any one of claims 20 and 27.

47. A method of treating atherosclerosis in a mammal, the method comprising administering to the mammal a dosage form from any one of claims 20 and 27.

48. A method of treating hypotension in a mammal, the method comprising administering to the mammal a dosage form from any one of claims 20 and 27.

49. A composition comprising:
from about 20% to about 40% by weight of a co-solvent;
from about 4% to about 9% by weight of a pH modifier;
from about 9% to about 18% by weight of purified water;
from about 28% to about 56% by weight of a surfactant; and
from about 5% to about 10% by weight of a weakly ionizable drug.

50. A composition comprising:
from about 10% to about 20% by weight of a co-solvent;
from about 2% to about 5% by weight of a pH modifier;
from about 4% to about 9% by weight of purified water;
from about 14% to about 28% by weight of a surfactant;
from about 2% to about 6% by weight of a weakly ionizable drug; and
from about 35% to about 65% by weight of a solid particle adsorbent.

51. A composition comprising:
from about 20% to about 40% by weight of a co-solvent;
from about 4% to about 9% by weight of a pH modifier;
from about 9% to about 18% by weight of purified water;
from about 28% to about 56% by weight of a surfactant; and
from about 5% to about 10% by weight of meloxicam.

52. The composition of claim 51, the composition comprising:
about 30% by weight of a co-solvent;
about 6.67% by weight of a pH modifier;
about 13.33% by weight of purified water;
about 42% by weight of a surfactant; and
about 8% by weight of meloxicam.

53. The composition of claim 52, the composition comprising:
about 3% by weight of Captex 355®;
about 5% by weight Capmul MCM®;
about 2% by weight PVP K12;
about 20% by weight Propylene Glycol;
about 6.67% by weight aqueous tris(hydroxymethyl)aminomethane;
about 13.33% by weight purified water;
about 42% by weight Tween 80®; and
about 8% by weight meloxicam.
